SEQUENCE LISTING (59 sequences)

SEQ ID NO: 1 - protein, 194 aa, Homo sapiens
MEEKLKKTKIIFVVGGPGSGKGTQCEKIVQKYGYTHLSTGDLLRSEVSSGSARGKKLSEIMEKGQLVPLETVLDMLRDAMVAKVNTSKGFLIDGYPREVQQGEEFERRIGQPTLLLYVDAGPETMTQRLLKRGETSGRVDDNEETIKKRLETYYKATEPVIAFYEKRGIVRKVNAEGSVDSVFSQVCTHLDALK

SEQ ID NO: 2 - protein, 239 aa, Homo sapiens
MAPSVPAAEPEYPKGIRAVLLGPPGAGKGTQAPRLAENFCVCHLATGDMLRAMVASGSELGKKLKATMDAGKLVSDEMVVELIEKNLETPLCKNGFLLDGFPRTVRQAEMLDDLMEKRKEKLDSVIEFSIPDSLLIRRITGRLIHPKSGRSYHEEFNPPKEPMKDDITGEPLIRRSDDNEKALKIRLQAYHTQTTPLIEYYRKRGIHSAIDASQTPDVVFASILAAFSKATCKDLVMFI

SEQ ID NO: 3 - protein, 227 aa, Homo sapiens
MGASARLLRAVIMGAPGSGKGTVSSRITTHFELKHLSSGDLLRDNMLRGTEIGVLAKAFIDQGKLIPDDVMTRLALHELKNLTQYSWLLDGFPRTLPQAEALDRAYQIDTVINLNVPFEVIKQRLTARWIHPASGRVYNIEFNPPKTVGIDDLTGEPLIQREDDKPETVIKRLKAYEDQTKPVLEYYQKKGVLETFSGTETNKIWPYVYAFLQTKVPQRSQKASVTP

SEQ ID NO: 4 - protein, 223 aa, Homo sapiens
MASKLLRAVILGPPGSGKGTVCQRIAQNFGLQHLSSGHFLRENIKASTEVGEMAKQYIEKSLLVPDHVITRLMMSELENRRGQHWLLDGFPRTLGQAEALDKICEVDLVISLNIPFETLKDRLSRRWIHPPSGRVYNLDFNPPHVHGIDDVTGEPLVQQEDDKPEAVAARLRQYKDVAKPVIELYKSRGVLHQFSGTETNKIWPYVYTLFSNKITPIQSKEAY

SEQ ID NO: 5 - protein, 198 aa, Homo sapiens [sequence omitted]

SEQ ID NO: 6 - protein, 172 aa, Homo sapiens
MLLPNILLTGTPGVGKTTLGKELASKSGLKYINVGDLAREEQLYDGYDEEYDCPILDEDRVVDELDNQMREGGVIVDYHGCDFFPERWFHIVFVLRTDTNVLYERLETRGYNEKKLTDNIQCEIFQVLYEEATASYKEEIVHQLPSNKPEELENNVDQILKWIEQWIKDHNS

SEQ ID NO: 7 - protein, 173 aa, Escherichia coli
MAEKRNIFLVGPMGAGKSTIGRQLAQQLNMEFYDSDQEIEKRTGADVGWVFDLEGEEGFRDREEKVINELTEKQGIVLATGGGSVKSRETRNRLSARGVVVYLETTIEKQLARTQRDKKRPLLHVETPPREVLEALANERNPLYEEIADVTIRTDDQSAKVVANQIIHMLESN

SEQ ID NO: 8 - protein, 174 aa, Escherichia coli
MTQPLFLIGPRGCGKTTVGMALADSLNRRFVDTDQWLQSQLNMTVAEIVEREEWAGFRARETAALEAVTAPSTVIATGGGIILTEFNRHFMQNNGIVVYLCAPVSVLVNRLQAAPEEDLRPTLTGKPLSEEVQEVLEERDALYREVAHIIIDATNEPSQVISEIRSALAQTINC

SEQ ID NO: 9 - protein, 173 aa, Klebsiella pneumoniae
MAEKRNIFLVGPMGAGKSTIGRQLAQQLNMEFYDSDQEIEKRTGADVGWVFDVEGEEGFRDREEKIINELTEKQGIVLATGGGSVKSRETRNRLSARGVVVYLETTIEKQLARTQRDKKRPLLQVDAPPREVLEALADERNPLYEEIADVTIRTDDQSAKVVANQIIHMLESN

SEQ ID NO: 10 - protein, 177 aa, Klebsiella pneumoniae
MTQPIFLIGPRGCGKTTVGHALARARHFQFSDTDHRLQAHEQRTVAEIVQAEGWARFRELETLSLKAVTLPNTVIATGGGIVLAEGNRQFMRENGVVIYLQASVSALIDRLEAYPKAEQRPTLTGKPVREEVGEVLAQREALYRDAAHHIVDATASPDRVVEQIMSMLCSATATPVS

SEQ ID NO: 11 - protein, 173 aa, Yersinia pestis
MAEKRNIFLVGPMGAGKSTIGRQLAQQLNMEFFDSDQEIERRTGADVGWVFDVEGEEGFRDREEKVINELTEKQGIVLATGGGSVKSRETRNRLSARGVVVYLETTIEKQLARTQRDKKRPLLQVDEPPREVLEALAKERNPLYEEIADVTIRTDDQSAKVVANQIINMLESN

SEQ ID NO: 12 - protein, 174 aa, Yersinia pestis
MTQTIFMVGARGAGKTTIGKALAQALGYRFVDTDLFMQQTSQMTVAEVVESEGWDGFRLRESMALQAVTAPKTVVATGGGAVLSSENRAFMRDHGRVIYLRASAAVLAKRLAEDPEEAQRPSLTGKPIVEEILDVLASREALYQDVAHHVLDGTQTPSLVVEQILQMLTGEMVK

SEQ ID NO: 13 - protein, 173 aa, Shigella flexneri; identical to SEQ ID NO: 7

SEQ ID NO: 14 - protein, 174 aa, Shigella flexneri
MTQPLFLIGPRGCGKTTVGMALADSLNRRFVDTDQWLQSQLNMTVAEIVEREEWAGFRARETAALEAVTAASTVIATGGGIILTEFNRHFMQNNGIVVYLCAPVSVLVNRLQAAPEEDLRPTLTGKPLSEEVQEVLEERDALYREVAHIIIDATNEPSQVISEIRSALAQTINC

SEQ ID NO: 15 - protein, 176 aa, Mycobacterium tuberculosis
MAPKAVLVGLPGSGKSTIGRRLAKALGVGLLDTDVAIEQRTGRSIADIFATDGEQEFRRIEEDVVRAALADHDGVLSLGGGAVTSPGVRAALAGHTVVYLEISAAEGVRRTGGNTVRPLLAGPDRAEKYRALMAKRAPLYRRVATMRVDTNRRNPGAVVRHILSRLQVPSPSEAAT

SEQ ID NO: 16 - protein, 574 aa, Rattus Norvegicus
MSVQENTLPQQLWPWIFRSQKDLAKSALSGAPGGPAGYLRRASVAQLTQELGTAFFQQQQLPAAMADTFLEHLCLLDIDSQPVAARSTSIIATIGPASRSVDRLKEMIKAGMNIARLNFSHGSHEYHAESIANIREATESFATSPLSYRPVAIALDTKGPEIRTGVLQGGPESEVEIVKGSQVLVTVDPKFQTRGDAKTVWVDYHNITRVVAVGGRIYIDDGLISLVVQKIGPEGLVTEVEHGGILGSRKGVNLPNTEVDLPGLSEQDLLDLRFGVQHNVDIIFASFVRKASDVLAVRDALGPEGQNIKIISKIENHEGVKKFDEILEVSDGIMVARGDLGIEIPAEKVFLAQKMMIGRCNLAGKPVVCATQMLESMITKARPTRAETSDVANAVLDGADCIMLSGETAKGSFPVEAVMMQHAIAREAEAAVYHRQLFEELRRAAPLSRDPTEVTAIGAVEASFKCCAAAIIVLTKTGRSAQLLSQYRPRAAVIAVTRSAQAARQVHLSRGVFPLLYREPPEAIWADDVDRRVQFGIESGKLRGFLRVGDLVIVVTGWRPGSGYTNIMRVLSVS

SEQ ID NO: 17 - protein, 543 aa, Rattus norvegicus
MEGPAGYLRRASVAQLTQELGTAFFQQQQLPAAMADTFLEHLCLLDIDSQPVAARSTSIIATIGPASRSVDRLKEMIKAGMNIARLNFSHGSHEYHAESIANIREATESFATSPLSYRPVAIALDTKGPEIRTGVLQGGPESEVEIVKGSQVLVTVDPKFQTRGDAKTVWVDYHNITRVVAVGGRIYIDDGLISLVVQKIGPEGLVTEVEHGGILGSRKGVNLPNTEVDLPGLSEQDLLDLRFGVQHNVDIIFASFVRKASDVLAVRDALGPEGQNIKIISKIENHEGVKKFDEILEVSDGIMVARGDLGIEIPAEKVFLAQKMMIGRCNLAGKPVVCATQMLESMITKARPTRAETSDVANAVLDGADCIMLSGETAKGSFPVEAVMMQHAIAREAEAAVYHRQLFEELRRAAPLSRDPTEVTAIGAVEASFKCCAAAIIVLTKTGRSAQLLSQYRPRAAVIAVTRSAQAARQVHLSRGVFPLLYREPPEAIWADDVDRRVQFGIESGKLRGFLRVGDLVIVVTGWRPGSGYTNIMRVLSVS

SEQ ID NO: 18 - protein, 531 aa, Rattus norvegicus
MSKSHSEAGSAFIQTQQLHAAMADTFLEHMCRLDIDSAPITARNTGIICTIGPASRSVETLKEMIKSGMNVARMNFSHGTHEYHAETIKNVRTATESFASDPILYRPVAVALDTKGPEIRTGLIKGSGTAEVELKKGATLKITLDNAYMEKCDENILWLDYKNICKVVDVGSKVYVDDGLISLQVKQKGPDFLVTEVENGGFLGSKKGVNLPGAAVDLPAVSEKDIQDLKFGVEQDVDMVFASFIRKAADVHEVRKILGEKGKNIKIISKIENHEGVRRFDEILEASDGIMVARGDLGIEIPAEKVFLAQKMIIGRCNRAGKPVICATQMLESMIKKPRPTRAEGSDVANAVLDGADCIMLSGETAKGDYPLEAVRMQHLIAREAEAAMFHRKLFEELARSSSHSTDLMEAMAMGSVEASYKCLAAALIVLTESGRSAHQVARYRPRAPIIAVTRNHQTARQAHLYRGIFPVVCKDPVQEAWAEDVDLRVNLAMNVGKARGFFKKGDVVIVLTGWRPGSGFTNTMRVVPVP

SEQ ID NO: 19 - protein, 531 aa, Rattus norvegicus
MPKPDSEAGTAFIQTQQLHAAMADTFLEHMCRLDIDSAPITARNTGIICTIGPASRSVEMLKEMIKSGMNVARLNFSHGTHEYHAETIKNVRAATESFASDPILYRPVAVALDTKGPEIRTGLIKGSGTAEVELKKGATLKITLDNAYMEKCDENILWLDYKNICKVVEVGSKIYVDDGLISLQVKEKGADYLVTEVENGGSLGSKKGVNLPGAAVDLPAVSEKDIQDLKFGVEQDVDMVFASFIRKAADVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAVFHRLLFEELARASSQSTDPLEAMAMGSVEASYKCLAAALIVLTESGRSAHQVARYRPRAPIIAVTRNPQTARQAHLYRGIFPVLCKDAVLDAWAEDVDLRVNLAMNVGKARGFFKKGDVVIVLTGWRPGSGFTNTMRVVPVP

SEQ ID NO: 20 - protein, 381 aa, Homo sapiens
MPFSNSHNALKLRFPAEDEFPDLSAHNNHMAKVLTPELYAELRAKSTPSGFTLDDVIQTGVDNPGHPYIMTVGCVAGDEESYEVFKDLFDPIIEDRHGGYKPSDEHKTDLNPDNLQGGDDLDPNYVLSSRVRTGRSIRGFCLPPHCSRGERRAIEKLAVEALSSLDGDLAGRYYALKSMTEAEQQQLIDDHFLFDKPVSPLLLASGMARDWPDARGIWHNDNKTFLVWVNEEDHLRVISMQKGGNMKEVFTRFCTGLTQIETLFKSKDYEFMWNPHLGYILTCPSNLGTGLRAGVHIKLPNLGKHEKFSEVLKRLRLQKRGTGGVDTAAVGGVFDVSNADRLGFSEVELVQMVVDGVKLLIEMEQRLEQGQAIDDLMPAQK

SEQ ID NO: 21 - protein, 381 aa, Homo sapiens
MPFGNTHNKFKLNYKPEEEYPDLSKHNNHMAKVLTLELYKKLRDKETPSGFTVDDVIQTGVDNPGHPFIMTVGCVAGDEESYEVFKELFDPIISDRHGGYKPTDKHKTDLNHENLKGGDDLDPNYVLSSRVRTGRSIKGYTLPPHCSRGERRAVEKLSVEALNSLTGEFKGKYYPLKSMTEKEQQQLIDDHFLFDKPVSPLLLASGMARDWPDARGIWHNDNKSFLVWVNEEDHLRVISMEKGGNMKEVFRRFCVGLQKIEEIFKKAGHPFMWNQHLGYVLTCPSNLGTGLRGGVHVKLAHLSKHPKFEEILTRLRLQKRGTGGVDTAAVGSVFDVSNADRLGSSEVEQVQLVVDGVKLMVEMEKKLEKGQSIDDMIPAQK

SEQ ID NO: 22 - protein, 417 aa, Homo sapiens
MAGPFSRLLSARPGLRLLALAGAGSLAAGFLLRPEPVRAASERRRLYPPSAEYPDLRKHNNCMASHLTPAVYARLCDKTTPTGWTLDQCIQTGVDNPGHPFIKTVGMVAGDEETYEVFADLFDPVIQERHNGYDPRTMKHTTDLDASKIRSGYFDERYVLSSRVRTGRSIRGLSLPPACTRAERREVERVVVDALSGLKGDLAGRYYRLSEMTEAEQQQLIDDHFLFDKPVSPLLTAAGMARDWPDARGIWHNNEKSFLIWVNEEDHTRVISMEKGGNMKRVFERFCRGLKEVERLIQERGWEFMWNERLGYILTCPSNLGTGLRAGVHIKLPLLSKDSRFPKILENLRLQKRGTGGVDTAATGGVFDISNLDRLGKSEVELVQLVIDGVNYLIDCERRLERGQDIRIPTPVIHTKH

SEQ ID NO: 23 - protein, 419 aa, Homo sapiens
MASIFSKLLTGRNASLLFATMGTSVLTTGYLLNRQKVCAEVREQPRLFPPSADYPDLRKHNNCMAECLTPAIYAKLRNKVTPNGYTLDQCIQTGVDNPGHPFIKTVGMVAGDEESYEVFADLFDPVIKLRHNGYDPRVMKHTTDLDASKITQGQFDEHYVLSSRVRTGRSIRGLSLPPACTRAERREVENVAITALEGLKGDLAGRYYKLSEMTEQDQQRLIDDHFLFDKPVSPLLTCAGMARDWPDARGIWHNYDKTFLIWINEEDHTRVISMEKGGNMKRVFERFCRGLKEVERLIQERGWEFMWNERLGYILTCPSNLGTGLRAGVHVRIPKLSKDPRFSKILENLRLQKRGTGGVDTAAVADVYDISNIDRIGRSEVELVQIVIDGVNYLVDCEKKLERGQDIKVPPPLPQFGKK

SEQ ID NO: 24 - protein, 523 aa, Mus musculus
MAAALQVLPCLLRAPSRPLLWGPPVARMTSGMALAEQARQLFDSAVGAVQPGPMLQRTLSLDPSGRQLKVRDRTFQLRENLYLVGFGKAVLGMAAAAEELLAQHLVQGVISVPKGIRAAMEHAGKKEMLLKPHSRVQVFEGAEDNLPDRDALRAALTIQQLAEGLTADDLLLVLISGGGSALLPAPIPPVTLEEKQMLTKLLAARGATIQELNTIRKALSQLKGGGLAQAAYPAQVISLILSDVIGDPLEVIASGPTVASAHSVQDCLHILNHYGLRAALPRSVKTVLSRADSDPHGPHTCGHVLNVIIGSNSLALAEAQRQAEVLGYHAMVLSTAMQGDVKRVARFYGLLARVAAAHLTPSLAERPLEEEAELHQLAAELQLPDLQLEEALEAVAKAKGPVCLLAGGEPTVQLQGSGKGGRNQELALHVGVELGRQPLGPIDVLFLSGGTDGQDGPTKVAGAWVMSDLISQASAESLDIATSLTNNDSYTFFCRFRGGTHLLHTGLTGTNVMDVHLLILHPQ

SEQ ID NO: 25 - protein, 396 aa, Mus musculus
MLLKPHSRVQVFEGAEDNLPDRDALRAALTIQQLAEGLTADDLLLVLISGGGSALLPAPIPPVTLEEKQMLTKLLAARGATIQELNTIRKALSQLKGGGLAQAAYPAQVISLILSDVIGDPLEVIASGPTVASAHSVQDCLHILNHYGLRAALPRSVKTVLSRADSDPHGPHTCGHVLNVIIGSNSLALAEAQRQAEVLGYHAMVLSTAMQGDVKRVARFYGLLARVAAAHLTPSLAERPLEEEAELHQLAAELQLPDLQLEEALEAVAKAKGPVCLLAGGEPTVQLQGSGKGGRNQELALHVGVELGRQPLGPIDVLFLSGGTDGQDGPTKVAGAWVMSDLISQASAESLDIATSLTNNDSYTFFCRFRGGTHLLHTGLTGTNVMDVHLLILHPQ

SEQ ID NO: 26 - protein, 917 aa, Homo sapiens
MIAAQLLAYYFTELKDDQVKKIDKYLYAMRLSDETLIDIMTRFRKEMKNGLSRDFNPTATVKMLPTFVRSIPDGSEKGDFIALDLGGSSFRILRVQVNHEKNQNVHMESEVYDTPENIVHGSGSQLFDHVAECLGDFMEKRKIKDKKLPVGFTFSFPCQQSKIDEAILITWTKRFKASGVEGADVVKLLNKAIKKRGDYDANIVAVVNDTVGTMMTCGYDDQHCEVGLIIGTGTNACYMEELRHIDLVEGDEGRMCINTEWGAFGDDGSLEDIRTEFDREIDRGSLNPGKQLFEKMVSGMYLGELVRLILVKMAKEGLLFEGRITPELLTRGKFNTSDVSAIEKNKEGLHNAKEILTRLGVEPSDDDCVSVQHVCTIVSFRSANLVAATLGAILNRLRDNKGTPRLRTTVGVDGSLYKTHPQYSRRFHKTLRRLVPDSDVRFLLSESGSGKGAAMVTAVAYRLAEQHRQIEETLAHFHLTKDMLLEVKKRMRAEMELGLRKQTHNNAVVKMLPSFVRRTPDGTENGDFLALDLGGTNFRVLLVKIRSGKKRTVEMHNKIYAIPIEIMQGTGEELFDHIVSCISDFLDYMGIKGPRMPLGFTFSFPCQQTSLDAGILITWTKGFKATDCVGHDVVTLLRDAIKRREEFDLDVVAVVNDTVGTMMTCAYEEPTCEVGLIVGTGSNACYMEEMKNVEMVEGDQGQMCINMEWGAFGDNGCLDDIRTHYDRLVDEYSLNAGKQRYEKMISGMYLGEIVRNILIDFTKKGFLFRGQISETLKTRGIFETKFLSQIESDRLALLQVRAILQQLGLNSTCDDSILVKTVCGVVSRRAAQLCGAGMAAVVDKIRENRGLDRLNVTVGVDGTLYKLHPHFSRIMHQTVKELSPKCNVSFLLSEDGSGKGAALITAVGVRLRTEASS

SEQ ID NO: 27 - protein, 917 aa, Homo sapiens
MIASHLLAYFFTELNHDQVQKVDQYLYHMRLSDETLLEISKRFRKEMEKGLGATTHPTAAVKMLPTFVRSTPDGTEHGEFLALDLGGTNFRVLWVKVTDNGLQKVEMENQIYAIPEDIMRGSGTQLFDHIAECLANFMDKLQIKDKKLPLGFTFSFPCHQTKLDESFLVSWTKGFKSSGVEGRDVVALIRKAIQRRGDFDIDIVAVVNDTVGTMMTCGYDDHNCEIGLIVGTGSNACYMEEMRHIDMVEGDEGRMCINMEWGAFGDDGSLNDIRTEFDQEIDMGSLNPGKQLFEKMISGMYMGELVRLILVKMAKEELLFGGKLSPELLNTGRFETKDISDIEGEKDGIRKAREVLMRLGLDPTQEDCVATHRICQIVSTRSASLCAATLAAVLQRIKENKGEERLRSTIGVDGSVYKKHPHFAKRLHKTVRRLVPGCDVRFLRSEDGSGKGAAMVTAVAYRLADQHRARQKTLEHLQLSHDQLLEVKRRMKVEMERGLSKETHASAPVKMLPTYVCATPDGTEKGDFLALDLGGTNFRVLLVRVRNGKWGGVEMHNKIYAIPQEVMHGTGDELFDHIVQCIADFLEYMGMKGVSLPLGFTFSFPCQQNSLDESILLKWTKGFKASGCEGEDVVTLLKEAIHRREEFDLDVVAVVNDTVGTMMTCGFEDPHCEVGLIVGTGSNACYMEEMRNVELVEGEEGRMCVNMEWGAFGDNGCLDDFRTEFDVAVDELSLNPGKQRFEKMISGMYLGEIVRNILIDFTKRGLLFRGRISERLKTRGIFETKFLSQIESDCLALLQVRAILQHLGLESTCDDSIIVKEVCTVVARRAAQLCGAGMAAVVDRIRENRGLDALKVTVGVDGTLYKLHPHFAKVMHETVKDLAPKCDVSFLQSEDGSGKGAALITAVACRIREAGQR

SEQ ID NO: 28 - protein, 923 aa, Homo sapiens
MDSIGSSGLRQGEETLSCSEEGLPGPSDSSELVQECLQQFKVTRAQLQQIQASLLGSMEQALRGQASPAPAVRMLPTYVGSTPHGTEQGDFVVLELGATGASLRVLWVTLTGIEGHRVEPRSQEFVIPQEVMLGAGQQLFDFAAHCLSEFLDAQPVNKQGLQLGFSFSFPCHQTGLDRSTLISWTKGFRCSGVEGQDVVQLLRDAIRRQGAYNIDVVAVVNDTVGTMMGCEPGVRPCEVGLVVDTGTNACYMEEARHVAVLDEDRGRVCVSVEWGSFSDDGALGPVLTTFDHTLDHESLNPGAQRFEKMIGGLYLGELVRLVLAHLARCGVLFGGCTSPALLSQGSILLEHVAEMEDPSTGAARVHAILQDLGLSPGASDVELVQHVCAAVCTRAAQLCAAALAAVLSCLQHSREQQTLQVAVATGGRVCERHPRFCSVLQGTVMLLAPECDVSLIPSVDGGGRGVAMVTAVAARLAAHRRLLEETLAPFRLNHDQLAAVQAQMRKAMAKGLRGEASSLRMLPTFVRATPDGSERGDFLALDLGGTNFRVLLVRVTTGVQITSEIYSIPETVAQGSGQQLFDHIVDCIVDFQQKQGLSGQSLPLGFTFSFPCRQLGLDQGILLNWTKGFKASDCEGQDVVSLLREAITRRQAVELNVVAIVNDTVGTMMSCGYEDPRCEIGLIVGTGTNACYMEELRNVAGVPGDSGRMCINMEWGAFGDDGSLAMLSTRFDASVDQASINPGKQRFEKMISGMYLGEIVRHILLHLTSLGVLFRGQQIQRLQTRDIFKTKFLSEIESDSLALRQVRAILEDLGLPLTSDDALMVLEVCQAVSQRAAQLCGAGVAAVVEKIRENRGLEELAVSVGVDGTLYKLHPRFSSLVAATVRELAPRCVVTFLQSEDGSGKGAALVTAVACRLAQLTRV

SEQ ID NO: 29 - protein, 465 aa, Homo sapiens [sequence omitted]

SEQ ID NO: 30 - protein, 820 aa, Escherichia coli
MRVLKFGGTSVANAERFLRVADILESNARQGQVATVLSAPAKITNHLVAMIEKTISGQDALPNISDAERIFAELLTGLAAAQPGFPLAQLKTFVDQEFAQIKHVLHGISLLGQCPDSINAALICRGEKMSIAIMAGVLEARGHNVTVIDPVEKLLAVGHYLESTVDIAESTRRIAASRIPADHMVLMAGFTAGNEKGELVVLGRNGSDYSAAVLAACLRADCCEIWTDVDGVYTCDPRQVPDARLLKSMSYQEAMELSYFGAKVLHPRTITPIAQFQIPCLIKNTGNPQAPGTLIGASRDEDELPVKGISNLNNMAMFSVSGPGMKGMVGMAARVFAAMSRARISVVLITQSSSEYSISFCVPQSDCVRAERAMQEEFYLELKEGLLEPLAVTERLAIISVVGDGMRTLRGISAKFFAALARANINIVAIAQGSSERSISVVVNNDDATTGVRVTHQMLFNTDQVIEVFVIGVGGVGGALLEQLKRQQSWLKNKHIDLRVCGVANSKALLTNVHGLNLENWQEELAQAKEPFNLGRLIRLVKEYHLLNPVIVDCTSSQAVADQYADFLREGFHVVTPNKKANTSSMDYYHQLRYAAEKSRRKFLYDTNVGAGLPVIENLQNLLNAGDELMKFSGILSGSLSYIFGKLDEGMSFSEATTLAREMGYTEPDPRDDLSGMDVARKLLILARETGRELELADIEIEPVLPAEFNAEGDVAAFMANLSQLDDLFAARVAKARDEGKVLRYVGNIDEDGVCRVKIAEVDGNDPLFKVKNGENALAFYSHYYQPLPLVLRGYGAGNDVTAAGVFADLLRTLSWKLGV

SEQ ID NO: 31 - protein, 810 aa, Escherichia coli
MSVIAQAGAKGRQLHKFGGSSLADVKCYLRVAGIMAEYSQPDDMMVVSAAGSTTNQLINWLKLSQTDRLSAHQVQQTLRRYQCDLISGLLPAEEADSLISAFVSDLERLAALLDSGINDAVYAEVVGHGEVWSARLMSAVLNQQGLPAAWLDAREFLRAERAAQPQVDEGLSYPLLQQLLVQHPGKRLVVTGFISRNNAGETVLLGRNGSDYSATQIGALAGVSRVTIWSDVAGVYSADPRKVKDACLLPLLRLDEASELARLAAPVLHARTLQPVSGSEIDLQLRCSYTPDQGSTRIERVLASGTGARIVTSHDDVCLIEFQVPASQDFKLAHKEIDQILKRAQVRPLAVGVHNDRQLLQFCYTSEVADSALKILDEAGLPGELRLRQGLALVAMVGAGVTRNPLHCHRFWQQLKGQPVEFTWQSDDGISLVAVLRTGPTESLIQGLHQSVFRAEKRIGLVLFGKGNIGSRWLELFAREQSTLSARTGFEFVLAGVVDSRRSLLSYDGLDASRALAFFNDEAVEQDEESLFLWMRAHPYDDLVVLDVTASQQLADQYLDFASHGFHVISANKLAGASDSNKYRQIHDAFEKTGRHWLYNATVGAGLPINHTVRDLIDSGDTILSISGIFSGTLSWLFLQFDGSVPFTELVDQAWQQGLTEPDPRDDLSGKDVMRKLVILAREAGYNIEPDQVRVESLVPAHCEGGSIDHFFENGDELNEQMVQRLEAAREMGLVLRYVARFDANGKARVGVEAVREDHPLASLLPCDNVFAIESRWYRDNPLVIRGPGAGRDVTAGAIQSDINRLAQLL

SEQ ID NO: 32 - protein, 449 aa, Escherichia coli
MSEIVVSKFGGTSVADFDAMNRSADIVLSDANVRLVVLSASAGITNLLVALAEGLEPGERFEKLDAIRNIQFAILERLRYPNVIREEIERLLENITVLAEAAALATSPALTDELVSHGELMSTLLFVEILRERDVQAQWFDVRKVMRTNDRFGRAEPDIAALAELAALQLLPRLNEGLVITQGFIGSENKGRTTTLGRGGSDYTAALLAEALHASRVDIWTDVPGIYTTDPRVVSAAKRIDEIAFAEAAEMATFGAKVLHPATLLPAVRSDIPVFVGSSKDPRAGGTLVCNKTENPPLFRALALRRNQTLLTLHSLNMLHSRGFLAEVFGILARHNISVDLITTSEVSVALTLDTTGSTSTGDTLLTQSLLMELSALCRVEVEEGLALVALIGNDLSKACGVGKEVFGVLEPFNIRMICYGASSHNLCFLVPGEDAEQVVQKLHSNLFE

SEQ ID NO: 33 - protein, 468 aa, Salmonella typhimurium
SAEHVLTMLNEHEVKFVDLRFTDTKGKEQHVTIPAHQVNAEFFEEGKMFDGSSIGGWKGINESDMVLMPDASTAVIDPFFADSTLIIRCDILEPGTLQGYDRDPRSIAKRAEDYLRATGIADTVLFGPEPEFFLFDDIRFGASISGSHVAIDDIEGAWNSSTKYEGGNKGHRPGVKGGYFPVPPVDSAQDIRSEMCLVMEQMGLVVEAHHHEVATAGQNEVATRFNTMTKKADEIQIYKYVVHNVAHRFGKTATFMPKPMFGDNGSGMHCHMSLAKNGTNLFSGDKYAGLSEQALYYIGGVIKHAKAINALANPTTNSYKRLVPGYEAPVMLAYSARNRSASIRIPVVASPKARRIEVRFPDPAANPYLCFAALLMAGLDGIKNKIHPGEPMDKNLYDLPPEEAKEIPQVAGSLEEALNALDLDREFLKAGGVFTDEAIDAYIALRREEDDRVRMTPHPVEFELYYSV

SEQ ID NO: 34 - protein, 468 aa, Acidimicrobium ferrooxidans
MGYSPSDVVKLIQEKDIKFIDFRFTDTKGKEQHVSVPGHVIEEDTFTEGKAFDGSSIAGWKGINESDMILLPDPDSAVLDPFMDETTLLLRCDVIEPATGQGYERDPRSVAKRAEIYLKSTGIADTSFFGPEFFVFDSVTWNIDMSGCAYKVDAEEAAWNSGKEYESGNMGHRLGVKGGYFPVPPVDSAQDLRSAMCLAMEEMGLKVEVHHHEVATAGQHEIGVGFNTLTPRRMRCKILKYVVHNVAAVRQTATFMPKPVVGDNGSGMHVHQSLGKDGKNIFAGDLYGGLSEIALYYIGGIIKHAKAVNALTNPSTNSYKRLVPHFEAPVLLAYSAKNRSASIRIPYVNSPKARRIEVRFPDSTANPYLAFSAMLMAGLDGIQNKIHPATAMDKNLYDLPAEEQANIPGVAASLEEALRALEADHDFLMKGGVFSESWLEGYLDVKWAEVQTLRVTTHPVEFQMYYSL

SEQ ID NO: 35 - protein, 468 aa, Escherichia coli
SAEHVLTMLNEHEVKFVDLRFTDTKGKEQHVTIPAHQVNAEFFEEGKMFDGSSIGGWKGINESDMVLMPDASTAVIDPFFADSTLIIRCDILEPGTLQGYDRDPRSIAKRAEDYLRSTGIADTVLFGPEPEFFLFDDIRFGSSISGSHVAIDDIEGAWNSSTQYEGGNKGHRPAVKGGYFPVPPVDSAQDIRSEMCLVMEQMGLVVEAHHHEVATAGQNEVATRFNTMTKKADEIQIYKYVVHNVAHRFGKTATFMPKPMFGDNGSGMHCHMSLSKNGVNLFAGDKYAGLSEQALYYIGGVIKHAKAINALANPTTNSYKRLVPGYEAPVMLAYSARNRSASIRIPVVSSPKARRIEVRFPDPAANPYLCFAALLMAGLDGIKNKIHPGEAMDKNLYDLPPEEAKEIPQVAGSLEEALNELDLDREFLKAGGVFTDEAIDAYIALRREEDDRVRMTPHPVEFELYYSV

SEQ ID NO: 36 - protein, 491 aa, Archaeoglobus fulgidus
MVRRLRGDCMEEVERAKAVLKENNVRQVLCAFADVRGYLQMFSIPAREFVDGSAFENGIGFDGSSVRGFRTIEKSDMVWMPDASSLKIIPWIDDPIQKSAIMFGNVYEAWGTEIADCDPRGYVAKRYEDMLKSEGMSAIFGPEIEFFLFEGVDFTRLSWDMWVSPNGGAGDSWGPPRIMPISSELESGYMIRPKEGYFRPPPEDTTVEYRNELVYYLEQLGIDIEYHHHEVATAGQVELDFKPKQLVDVGDAFYLYKFAAKNIAAMHGLYATFMPKPLYLDNASGMHTHQSLWKGEPFSGEAVFADPDDEYMLSQKARYYIGGLLEHAKALTALCAPTVNSYKRLVPGFEAPIYICWSPRNRSALVRVPMYVKKPSAIRVEYRGVDPSCNPYLAITAQLAAGLDGIKKKIDPGDPLLEDVYELTPAQKRELGVGELPTTLRDAIDHLASDELMQEVLGSHIFDAFMELKIDEWNQYCLYITPWEFMKYFDI

SEQ ID NO: 37 - protein, 467 aa, Azotobacter vinelandii
MSKSLQLIKEHDVKWIDLRFTDTKGKQQHVTMPARDVDDDFFEYGKMFDGSSIAGWKGIEASDMILMPDDSTAVLDPFTEEPTLIIVCDIIEPSTMQGYDRDPRAIARRAEEYLKSTGIGDTAFFGPEPEFFIFDEVKYKSDISGSMFKIFSEQAAWNTDADFEGGNKGHRPGVKGGYFPVPPVDHDHEIRTAMCNALEEMGLKVEVHHHEVATAGQNEIGVSFNTLVAKADEVQTLKYCVHNVADAYGKTVTFMPKPLYGDNGSGMHVHMSIAKDGKNTFAGEGYAGLSDTALYFIGGIIKHGKALNGFTNPSTNSYKRLVPGFEAPVMLAYSARNRSASIRIPYVNSPKARRIEARFPDPSANPYLAFAALLMAGLDGIQNKIHPGDAADKNLYDLPPEEAKEIPQVCGSLKEALEELDKGRAFLTKGGVFSDDFIDAYLELKSEEEIKVRTFVHPLEYDLYYSV

SEQ ID NO: 38 - protein, 443 aa, Bacillus cereus
ARYTKEDIFRLAKEENVKYIRLQFTDLLGVIKNVEIPVSQLTKALDNKMMFDGSSIEGFVRIEESDMYLYPDLDTWVIFPWTAEKGKVARLICDIYNADGTPFEGDPRNNLKRVLKEMEALGFSDFNLGPEPEFFLFKVDEKGNPTLELNDNGGYFDLAPMDLGENCRRDIVLELEEMGFEIEASHHEVAPGQHEIDFKYANAIRSCDDIQTFKLVVKTIARKHGLHATFMPKPLYGVNGSGMHCNLSLFKNGENVFYDQNGDLQLSDDARHFIAGILKHAPAFTAVANPTVNSYKRLVPGYEAPCYVAWSAQNRSPLVRIPASRGISTRVEVRSVDPAANPYLVMATLLAAGLDGIKNKLTPPAAVDRNIYVMTKEEREEAGIVDLPATLAQALVTLQSNEVISNALGDHLLEHFIEAKEFEWDIFRTQVHQWERDQYMSLY

SEQ ID NO: 39 - protein, 443 aa, Bacillus subtilis
AKYTREDIEKLVKEENVKYIRLQFTDILGTIKNVEIPVSQLGKALDNKVMFDGSSIEGFVRIEESDMYLYPDLNTFVIFPWTAEKGKVARFICDIYNPDGTPFEGDPRNNLKRILKEMEDLGFSDFNLGPEPEFFLFKLDEKGEPTLELNDKGGYFDLAPTDLGENCRRDIVLELEEMGFEIEASHHEVAPGQHEIDFKYAGAVRSCDDIQTFKLVVKTIARKHGLHATFMPKPLFGVNGSGMHCNLSLFKNGVNAFFDENADLQLSETAKHFIAGIVKHATSFTAVTNPTVNSYKRLVPGYEAPCYVAWSAQNRSPLIRIPASRGISTRVEVRSVDPAANPYLALSVLLAAGLDGIKNKLEAPAPIDRNIYVMSKEERMENGIVDLPATLAEALEEFKSNEVMVKALGEHLFEHFIEAKEIEWDMFRTQVHPWEREQYMSQY

SEQ ID NO: 40 - protein, 454 aa, Halobacterium volcanii
MTEDNALTDGGLSDEAQAVIDEIEEKNVDFLRLQFTDILGTVKNVSIPASQAEKAFTEGIYFDGSSIDGFVRIQESDMRLEPDPSTFAVLPWRKKENSAAGRLICDVFNTSTGEPFSGDPRGVLKRAIEPEELGYDVNVAPEPEFFLFEEDEDGRATTVTNDAGGYFDLAPKDLASDVRRDIIYGLESMGFDIEASHHEVAEGQHEINFTYDDALSTADNVRTFRSVVRAIAAEHDLHATFMPKPIPRINGSGMHTHISLFKDGENAFHDGDDEFDLSDTAKSFVAGILDHAPAITASLTRRSTPTRRLVPGYEAPVYIAWSDRNRSALIRKPAARTPAASRIEARFPDPSCNPYLAFAALIHAGLDGVEKGLDCPDPVRENIYEFDEAKREEYGIETLPKTSAARRRPRRDEVIQEALGDHVFEKFVEAKRSEFKDYLVDVSQWELDRYLETF

SEQ ID NO: 41 - protein, 445 aa, Lactobacillus delbrueckii
MSKVITEEEIRKDVEEKNVRFLRLAFTDINGTLKNLEVPVSQLDDVLGNQTRFDGSSIDGFVRLEESDMVLYPDLATWLVLAWTTVEEGTIGRLVCSVHNVDGTPFEGDPRNNLKKVIAEMEEMGFSDFEIGFEAEFFLFKEGKNGEETTKVSDHSSYFDMASEDEGAKCRREIVETLEKLGFRVEAAHHEVGDGQQEIDFRFDNALATADKLQTFKMVVKTIARKYHLHASFMAKPVEGLAGNGMHTNMSLLKDGKNAFYDKDGQYNLSTTALTFLNGILEHARAITCVANPTVNSYKRLIPGFEAPVYISWASRNRSPMVRIPNANEVGTRLEMRSTDPTANPYLLLSACLKAGLTGIKEGKLPMAPVTSNLFEMTDDERKELGIKPLPSTLHNAIKAFKEDEVVKSAFSEHIVDSFLELKETEWALYTQSVSEWEVKRYFNY

SEQ ID NO: 42 - protein, 428 aa, Plasmodium falciparum
MCDIKRYNGFDYYKCPRTILKKTCEFVKNEGIADKVCIGNELEFFIFDKVNYSLDEYNTYLKVYDRESFSCKNDLSSIYGNHVVNKVEPHKDHFNNPNNEYLINDDSKKVKKKSGYFTTDPYDTSNIIKLRICRALNDMNINVQRYHHEVSTSQHEISLKYFDALTNADFLLITKQIIKTTVSSFNRTATFMPKPLVNDNGNGLHCNISLWKNNKNIFYHNDPSTFFLSKESFYFMYGIVKHAKALQAFCNATMNSYKRLVPGFETCQKLFYSFGSRSAVIRLSLINYSNPSEKRIEFRLPDCANSPHLVMAAIILAGYDGIKSKEQPLVPFESKDNHFYISSIFSKYVQHPENFNILTHALEGYESLHTINESPEFKNFFKCEEPQGISFSLVESLDALEKDHAFLTVNNIFTEVSQRIKNKHYHGK

SEQ ID NO: 43 - protein, 369 aa, Saccharomyces cerevisiae
AEASIEKTQILQKYLELDQRGRIIAEYVWIDGTGNLRSKGRTLKKRITSIDQLPEWNFDGSSTNQAPGHDSDIYLKPVAYYPDPFRRGDNIVVLAACYNNDGTPNKFNHRHEAAKLFAAHKDEEIWFGLEQEYTLFDMYDDVYGWPKGGYPAPQGPYYCGVGAGKVYARDMIEAHYRACLYAGLEISGINAEVMPSQWEFQVGPCTGIDMGDQLWMARYFLHRVAEEFGIKISFHPKPLKGDWNGAGCHTNVSTKEMRQPGGMKYIEQAIEKLSKRHAEHIKLYGSDNDMRLTGRHETASMTAFSSGVANRGSSIRIPRSVAKEGYGYFEDRRPASNIDPYLVTGIMCETVCGAIDNADMTKEFERESS

SEQ ID NO: 44 - protein, 382 aa, Chlamydomonas reinhardtii
MAAGSVGVFATDEKIGSLLDQSITRHFLSTVTDQQGKICAEYVWIGGSMHDVRSKSRTLSTIPTKPEDLPHWNYDGSSTGQAPGHDSEVYLIPRSIFKDPFRGGDNILVMCDCYEPPKVNPDGTLAAPKPIPTNTRFACAEVMEKAKKEEPWFGIEQEYTLLNAITKWPLGWPKGGYPAPQGPYYCSAGAGVAIGRDVAEVHYRLCLAAGVNISGVNAEVLPSQWEYQVGPCEGITMGDHMWMSRYIMYRVCEMFNVEVSFDPKPIPGDWNGSGGHTNYSTKATRTAPDGWKVIQEHCAKLEARHAVHIAAYGEGNERRLTGKHETSSMSDFSWGVANRGCSIRVGRMVPVEKSGYYEDRRPASNLDAYVVTRLIVETTILL

SEQ ID NO: 45 - protein, 357 aa, Zea mays
MASLTDLVNLDLSDCTDRIIAEYIWIGGTGIDLRSKARTVKGPITDPIQLPKWNYDGSSTGQAPGEDSEVILYPQAIFKDPFRKGNHILVMCDCYTPQGEPIPTNKRYSAAKVFSHPDVAAEVPWYGIEQEYTLLQKDVSWPLGWPVGGYPGPQGPYYCAAGADKAFGRDVVDAHYKACLYAGINISGINGEVMPGQWEFQVGPSVGISAGDEIWVARYILERITEMAGIVLSLDPKPIKGDWNGAGAHTNYSTKSMREAGGYEVIKAAIDKLGKRHKEHIAAYGEGNERRLTGRHETADINTFKWGVANRGASIRVGRDTEREGKGYFEDRRPASNMDPYVVTGMIAETTILWNGN

SEQ ID NO: 46 - protein, 357 aa, Orysa sativa
MANLTDLVNLNLSDCSDKIIAEYIWVGGSGIDLRSKARTVKGPITDVSQLPKWNYDGSSTGQAPGEDSEVILYPQAIFKDPFRRGDNILVMCDCYTPQGEPIPTNKRHSAAKIFSHPDVVAEVPWYGIEQEYTLLQKDVNWPLGWPVGGFPGPQGPYYCAAGAEKAFGRDIVDAHYKACIYAGINISGINGEVMPGQWEFQVGPSVGIAAADQVWVARYILERVTEVAGVVLSLDPKPIPGDWNGAGAHTNFSTKSMREPGGYEVIKKAIDKLALRHKEHIAAYGEGNERRLTGRHETADINTFKWGVANRGASIRVGRDTEKEGKGYFEDRRPASNMDPYVVTGMIAETTLLWKQN

SEQ ID NO: 47 - protein, 353 aa, Lupinus luteus
MSVLSDLINLNLSDTTEKIIAEYIWVGGSGVDLRSKARTLSGPVNDPSKLPKWNYDGSSTGQAPGKDSEVILWPQAIFKDPFRRGNNILVMCDTYTPAGEPIPTNKRHAAAKIFSHPDVVAEEPWFGIEQEYTLLQKDIHWPIGWPLGGFPGPQGPYYCGTGAEKAFGRDIVDSHYKACLYAGINISGINAEVMPGQWEFQVGPSVGISAGDELWVARYILERITEIAGVVLSLDPKPIPGDWNGAGAHTNYSTKSMRNDGGYEVIKKAIEKLGKRHNEHIAAYGEGNERRLTGRHETADISTFFWGVANRGASIRVGRDTEKEGKGYFEDRRPASNMDPYVVTSMIAETTLL

SEQ ID NO: 48 - protein, 357 aa, Pisum sativum
MSSLSDLINFNLSDSTEKIIAEYIWVGGSGIDIRSKARTLPGPVSDPAKLPKWNYDGSSTNQAPGKDSEVILYPQAIFKDPFRRGNNILVICDVYTPAGEPLPTNKRYNAAKIFSHPDVAAEVPWYGIEQEYTLLQKDINWPLGWPIGGYPGKQGPYYCGIGADKAYGRDIVDAHYKACLFAGINISGINGEVMPGQWEFQVGPSVGISAGDEIWAARYILERITEIAGVVVSFDPKPIPGDWNGAGAHANFSTKSMRENGGYEVIKKAIEKLGLRHKEHIAAYGEGNERRLTGKHETADINVFSWGVANRGSSIRVGRDTEKDGKGYFEDRRPASNMDPYVVTSMIAETTILWKKP

SEQ ID NO: 49 - protein, 399 aa, Drosophila melanogaster
MALRVAGLFLKKELVAPATQQLRLLRTGNTTRSQFLANSPNTALDKSILQRYRNLETPANRVQATYLWIDGTGENIRLKDRVLDKVPSSVEDLPDWQYDGSSTYQAHGENSDTTLKPRAIYRDPFKPGKNDVIVLCDTYSADGKPTASNKRAAFQAAIDLISDQEPWFGIEQEYTLLRRGRTSFGWPENGFPAPQGPYYCGVGADRVYARDLVEAHVVACLYAGIDFAGTNAEVMPAQWEFQIGPAGIKACDDLWVSRYILQRIAEEYGVVVTFDPKPMEGQWNGAGRHTNFSTKEMRADGGIKAIEEPIEKLSKRHERHIKAYYPKEGKDNERRLVGRLETSSIDKFSWGVANRAVSVRVPRGVATAGKGYLEDRRPSSNCDPYAVCNAIVQTCLLNE

SEQ ID NO: 50 - protein, 403 aa, Squalus acanthia
MRICRSFLFLVKKCGNITPTIWRNQHTYKMATSASANLSKIVKKNYMELPQDGKVQAMYIWIDGTGEAVRCKTRTLDNEPKSIAELPEWNFDGSSTYQSEGSNSDMYLVPSAMFRDPFRRDPNKLVLCEVLKYNRKPAESNLRHSCQKIMSMIANEYPWFGMEQEYTLLGTDGHPFGWPSNCFPGPQGPYYCGVGADKAYGRDIVEAHYRACLYAGIELSGTNAEVMAAQWEYQVGPCEGIQMGDHLWISRFILHRVCEDFGIIASFDPKPIPGNWNGAGCHTNFSTKAMRDDGGLKYIEDSIEKLGKRHQYHIRAYDPKGGLDNARALTGHHETSNINEFSAGVANRGASIRIPRSVGQDKKGYFEDRRPSANCDPYAVTEALVRTCLLDESGDKPIEYNKN

SEQ ID NO: 51 - protein, 392 aa, Xenopus laevis
MSVSHSSRLNKGVREQYMKLPQGEKVQVTYVWIDGTGEGVRCKTRTLDQEPKTIDEIPEWNFDGSSTHQAEGSNSDMYLIPVQMFRDPFCLDPNKLVMCEVLKYNRKSAETNLRHTCKKIMEMVNDHRPWFGMEQEYTLLGINGHPYGWPENGFPGPQGPYYCGVGADKVYGRDVVESHYKACLYAGIKICGTNAEVMPSQWEFQVGPCEGIDMGDHLWMARFILHRVCEDFGVVATLDPKPMTGNWNGAGCHTNYSTESMRVEGGLKHIEDAIEKLGKRHDYHICVYDPRGGKDNSRRLTGQHETSSIHEFSAGVANRGASIRIPRQVGQEGYGYFEDRRPAANCDPYAVTEALVRTTILNETGSETKDYKNGAGFSRAIGMASPRDAAVF

SEQ ID NO: 52 - protein, 373 aa, Gallus gallus
MATSASSHLSKAIKHMYMKLPQGEKVQAMYIWIDGTGEHLRCKTRTLDHEPKSLEDLPEWNFDGSSTFQAEGSNSDMYLRPAAMFRDPFRKDPNKLVLCEVFKYNRQSADTNLRHTCRRIMDMVSNQHPWFGMEQEYTLLGTDGHPFGWPSNCFPGPQGPYYCGVGADKAYGRDIVEAHYRACLYAGVKIGGTNAEVMPAQWEFQVGPCEGIEMGDHLWIARFILHRVCEDFGVIVSFDPKPIPGNWNGAGCHTNFSTKNMREDGGLKHIEEAIEKLSKRHQYHIRAYDPKGGLDNARRLTGFHETSSIHEFSAGVANRGASIRIPRNVGHEKKGYFEDRGPSANCDPYAVTEALVRTCLLNETGDEPFEYKN

SEQ ID NO: 53 - protein, 373 aa, Mus musculus
MATSASSHLNKGIKQMYMSLPQGEKVQAMYIWVDGTGEGLRCKTRTLDCEPKCVEELPEWNFDGSSTFQSEGSNSDMYLHPVAMFRDPFRRDPNKLVLCEVFKYNRKPAETNLRHICKRIMDMVSNQHPWFGMEQEYTLMGTDGHPFGWPSNGFPGPQGPYYCGVGADKAYGRDIVEAHYRACLYAGVKITGTNAEVMPAQWEFQIGPCEGIRMGDHLWIARFILHRVCEDFGVIATFDPKPIPGNWNVAGCHTNFSTKAMREENGLKCIEEAIDKLSKRHQYHIRAYDPKGGLDNARALTGFHETSNINDFSAGVANRGASIRIPRTVGQEKKGYFEDRRLRANCDPYAVTEAIVRTCLLNETGDEPFQYKN

SEQ ID NO: 54 - protein, 373 aa, Cricetulus griseus
MATSASSHLNKGIKQMYMSLPQGEKVQAMYIWVDGTGEGLRCKTRTLDCEPKCVEELPEWNFDGSSTFQSESSNSDMYLSPVAMFRDPFRKEPNKLVFCEVFKYNQKPAETNLRHTCKRIMDMVSNQHPWFGMEQEYTLLGTDGHPFGWPSDGFPGPQGLYYCGVGADKAYRRDIMEAHYRACLYAGVKITGTYAEVKHAQWEFQIGPCEGIRMGDHLWVARFILHRVCKDFGVIATFDSKPIPGNWNGAGCHTNFSTKTMREENGLKHIKEAIEKLSKRHRYHIRAYDPKGGLDNARRLTGFHKTSNINDFSAGVADRSASIRIPRTVGQEKKGYFEARCPSANCDPFAVTEAIVRTCLLNETGDQPFQYKN

SEQ ID NO: 55 - protein, 373 aa, Homo sapiens
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 56 - DNA, 80 bp, Synthetic oligo
gaggatcccagaaccagcgccatcagcgttaccatggcaccagctacaaccttgaaccaattaaccctcactaaagggcg

SEQ ID NO: 57 - DNA, 81 bp, Synthetic oligo
gtggatccgcgatatcgtgaaacagcgcggcgatgaaaatcagctcagttgacggcagtaatacgactcactatagggctc

SEQ ID NO: 58 - DNA, 80 bp, Synthetic oligo
gaggatcctgcgcctgtttgaactgacgcagcgcctcaagctgttgctcttcgtcatcaattaaccctcactaaagggcg

SEQ ID NO: 59 - DNA, 81 bp, Synthetic oligo
gtggatccaggtgttccagctcattcgcggcggacgcgaaccgtcgctgcaatcgcgctaatacgactcactatagggctc

(19) United States
(12) Patent Application Publication
(10) Pub. No.: US 2012/0270251 A1
(43) Pub. Date: Oct. 25, 2012

(54) INHIBITION OF THE ACTIVITY OF KINASE AND SYNTHETASE ENZYMES

(75) Inventors: Robyn Roth, Sandton (ZA); Colin Peter Kenyon, Randburg (ZA)

(73) Assignee: CSIR, Pretoria (ZA)

(21) Appl. No.: 13/518,072

(22) PCT Filed: Dec. 22, 2010
(86) PCT No.: PCT/IB10/56006

§ 371 Date: Jun. 21, 2012

(30) Foreign Application Priority Data
Dec. 22, 2009 (ZA) 2009/09160

(51) Int. Cl. C12N 9/99; G06G 7/48; C12Q 1/50; C12Q 1/48; C12Q 1/25

(52) U.S. Cl. 435/17; 435/184; 435/15; 435/4; 703/11

(57) ABSTRACT

The invention provides a method of inhibiting the activity of a kinase or a synthetase, the method including binding an active site of the kinase or synthetase with a deuterated imidazole moiety, thereby inhibiting the activity of the kinase or the synthetase.

THIS INVENTION relates to the inhibition of the activity of kinase and synthetase enzymes. More particularly, the invention relates to a method of inhibiting the activity of a kinase or a synthetase, to a method of coupling a kinase or a synthetase to a nucleotide, to a method of generating a compound that inhibits the activity of a kinase or a synthetase, to a computer-assisted method of generating a test inhibitor of the activity of a kinase or a synthetase, and to a method of screening a test compound in vitro to determine whether or not it inhibits the activity of a kinase or a synthetase.

Adenylate kinase (AK) contributes to the homeostasis of adenine nucleotides by maintaining intracellular nucleotide pools. Six isoenzymes of adenylate kinase have been identified in mammalian cells with different subcellular localization and substrate specificity. Adenylate kinase catalyses the reaction:

ATP+AMP→2ADP

where ATP is adenosine triphosphate, AMT is adenosine monophosphate and ADP is adenosine diphosphate. The adenylate kinases (ATP:AMP phosphotransferases, EC 2.7.4.3) catalyze the reversible transfer of the γ-phosphate group from a phosphate donor (ATP, GTP, CTP, ITP) to AMP.

The phosphate donor is usually ATP. There is a size variation among the isoenzymes: AK1, AK5 and AK6 are short type adenylate kinases while AK2, AK3 and AK4 are long type adenylate kinases containing a 27 amino acid insertion sequence in the central portion of the peptide. The mammalian adenylate kinases have a distinct intracellular compartmentalization, with AK1 in the cytosol, AK2 in the inter-membrane space of mitochondria, AK3 in the mitochondrial matrix, AK4 being mitochondrial in nature, AK5 (unknown localization) and AK6 in the nucleus. AK3 present in the mitochondrial matrix has a preference for GTP over ATP.

It is often desired to regulate the activity of kinases and synthetases, and this can, for example, be done by binding an active site of a kinase, such as AK, or a synthetase, such as glutamine synthetase, to an imidazole moiety, eg as found in ATP. An object of this invention is to provide a means whereby the level of inhibition of the activity of a kinase or a synthetase can be enhanced.

Thus, according to a first aspect of the invention, there is provided a method of inhibiting the activity of a kinase or a synthetase, the method including binding an active site of the kinase or synthetase with a deuterated imidazole moiety, thereby inhibiting the activity of the kinase or the synthetase.

The method may be applied to a kinase. Examples of suitable kinases are adenylate kinase (AK), shikimate kinase (SK), pyruvate kinase (PK), hexokinase (HXK), aspartokinase (ASK), creatine kinase (CK), glycerate kinase, acetate kinase, glutamine synthetase, such as adenylated glutamineate synthetase, and/or deadenylated glutamine synthetase, phosphofructokinase, and isoforms thereof. In a particular embodiment of the invention, the kinase may be adenylate kinase (AK). Its sequence comprises conserved residues Arg 97, Glu 98, Arg 128 and Asp 180.

Instead, the method may be applied to a synthetase. The synthetase may then, for example, be glutamine synthetase (GS).

Generally, the kinase or the synthetase, or an isoenzyme thereof, may comprise amino acid residues identical to, or similar to, conserved adenylate kinase residues Arg 97, Glu 98, Arg 128 and Asp 180, at positions equivalent to the positions of these residues in adenylate kinase.

The inhibition of the kinase or the synthetase activity may be effected in vitro or in vivo.

More particularly, the deuterated imidazole moiety may be provided by a nucleotide. In one embodiment of the invention, the nucleotide may be adenosine triphosphate (ATP), adenosine diphosphate (ADP) or adenosine monophosphate (AMP), with the nucleotide having an immonium moiety induced at position N7, so that a carbene is induced at the C8 position, and with deuteration being effected at the C8 position. However, in another embodiment of the invention, the nucleotide may be a compound which is deuterated at a position equivalent to C8 in ATP, ADP and AMP.

The extent of regulation of each form (isoenzymes) of the kinase enzyme or the synthetase enzyme is different based on the level of the Kinetic Isotope Effect (KIE) and the kinetics of the enzymes and the structure of the active site of the kinase. When the kinase is adenylate kinase, the reaction catalysed by adenylate kinase is:

ATP+AMP→2ADP

The assay is carried out in the presence of ATP and AMP measuring the formation of ADP. The KIE is obtained when comparing the enzyme activity of the adenylate kinase enzyme when the reaction is carried out in the presence of ATP and deuterated ATP. A kinetic isotope effect is also obtained when comparing the activity of the enzyme when the reaction is carried out in the presence of AMP and deuterated AMP. A further KIE is obtained when both deuterated ATP and deuterated AMP are used in the reaction. It is significant that for all these enzymes, the KIE occurs as a result of the change in using ATP over deuterated ATP or AMP over deuterated AMP. The KIE is obtained by dividing the activity of the enzyme in the presence of the proteated species (v_{H}) by the enzyme activity in the presence of the deuterated species (v_{D}) and is v_{H}/v_{D}=0.5. The KIE is found over a broad range of ATP concentration enzyme activity profile. The extent of the regulation of AK is also ATP concentration dependent. The proposed basic reaction mechanisms by which all forms of kinase are regulated occurs via either the formation of an immonium species at N7, which in turn induces the formation of a carbene at C8 or via the delocalization of electrons away from the C8 rendering the C8 more acidic. An immonium species may be induced at N7 by (1) protonation via a coordinated HCO₃⁻, (2) carboxylation of the N7, or (3) the coordination of an amino acid side chain within the active site such as a arginine, glutamine, lysine or histidine. The different forms of regulation are then based on the mechanism by which the carbene formed at C8 is stabilized and the mechanism by which the C8-H is deprotonated or via the interaction of the protein coordinated amino acid side chains affecting the delocalization of electrons around the adenyl ring of the nucleotide.

The formation of the immonium species at N7 then facilitates the deprotonation of C8 via a coordinated amino acid residue. The resulting carbene intermediate is stabilised by the putative bond formation by a coordinated amino acid and C8. In some kinases the coordination may be mediated by the presence of a divalent metal ion such as Mn²⁺ coordinated into the ATP coordination complex.

In particular, it was found that the activity of AK1 is affected by the deuteration of ATP and AMP at the C8 position. The role of deuteration of ATP/AMP in causing a KIE in AK1 demonstrates that the binding mechanism of nucleotides to the active sites of kinases is similar and the imidazole moiety is implicated in all cases. “Imidazole” moiety-containing compounds are found to affect the regulation of AK1 and when these compounds are deuterated at the position in the imidazole moiety equivalent to the C8 position in ATP.

Thus, in accordance with the invention, the nucleotides ATP, ADP and AMP will be bound to the AK active site, with the nucleotide being either protonated or unprotonated at position N7. The protonated form is positively charged while the unprotonated form is neutral. The unprotonated from may be protonated within the active site of the enzyme also inducing the formation of an immonium species. In both cases, ie when the nucleotide is in either the protonated form or the neutral form, an immonium species at the N7 position facilitates the induction of a carbene at the C8 position.

In the case of the neutral form of the nucleotide, the method may include creating an immonium species at position N7 through the donation of a protein by the AK enzyme. This facilitates the induction of the carbene at the C8 position by making the C8-H more acidic.

The rendering of the C8-H to become more acidic via the coordination of amino acid side chains and the delocalization of electrons away from C8 is another mechanism by which the regulation of kinases occurs.

According to a second aspect of the invention, there is provided a method of coupling a kinase or a synthetase to a nucleotide or to a nucleotide analogue, to inhibit the activity of the kinase or the synthetase, the method including binding an active site of the kinase or the synthetase with a nucleotide or with a nucleotide analogue comprising an imidazole moiety in deuterated form.

According to a third aspect of the invention, there is provided a method of generating a compound that inhibits the activity of a kinase or a synthetase, the method comprising providing a three-dimensional structure of a kinase or a synthetase; and designing, based on the three-dimensional structure, a compound capable of inhibiting the activity of the kinase or the synthetase, the compound comprising a deuterated imidazole moiety.

The compound may be a nucleotide or imidazole containing compound as hereinbefore described.

According to a fourth aspect of the invention, there is provided a computer-assisted method of generating a test inhibitor of the activity of a kinase or a synthetase, the method using a processor and an input device, the method comprising

(a) inputting, on the input device, data comprising a structure of a kinase or a synthetase;

(b) docking into an active site of the kinase or the synthetase, a test inhibitor molecule comprising a deuterated imidazole moiety, using the processor; and

(c) determining, based on the docking, whether the test inhibitor compound would inhibit the kinase or synthetase activity.

The method may include determining, based on the docking, whether the test inhibitor molecule would inhibit the transfer of a y-phosphate group from a phosphate donor.

The method may further comprise designing a test inhibitor determined by step (c) to inhibit the kinase or the synthetase activity and evaluating the inhibitory activity of the test inhibitor on a bacterial kinase or synthetase in vitro.

According to a fifth aspect of the invention, there is provided a method of screening a compound in vitro to determine whether or not it inhibits the activity of kinase or a synthetase, the method comprising contacting a kinase or a synthetase with a compound comprising a protonated imidazole moiety; contacting the kinase or the synthetase with the same compound comprising a deuterated imidazole moiety; and determining whether or not the activity of the kinase or synthetase is reduced in the presence of the compound containing the deuterated imidazole moiety relative to the activity of the same kinase or synthetase in the presence of the compound containing the protonated imidazole moiety.

The method of the second, third, fourth or fifth aspect may be applied to a kinase. Examples of suitable kinases are then, as hereinbefore described, adenylate kinase (AK), shikimate kinase (SK), pyruvate kinase (PK), hexokinase (HXK), aspartokinase (ASK), creatine kinase (CK), glycerate kinase, acetate kinase, glutamine synthetase, such as adenylated glutamine synthetase, and/or deadenylated glutamine synthetase, phosphofructokinase, and isoforms thereof. Instead, the method of the second, third, fourth or fifth aspect of the invention may be applied to a synthetase. The synthetase may then, for example, be glutamine synthetase (GC).

As also hereinbefore described, the deuterated imidazole moiety may be provided by a nucleotide or by a nucleotide analogue. The nucleotide may, in one embodiment of the invention be adenosine triphosphate (ATP), adenosine diphosphate (ADP) or adenosine monophosphate (AMP), with the nucleotide having an immonium moiety at position N7, so that a carbene is induced at the C8 position, and with deuteration being effected at the C8 position. However, in another embodiment of the invention, the nucleotide may be a compound which is deuterated at a position equivalent to C8 in ATP, ADP and AMP.

The invention will now be described in more detail with reference to the accompanying non-limiting examples and drawings.

In the drawings

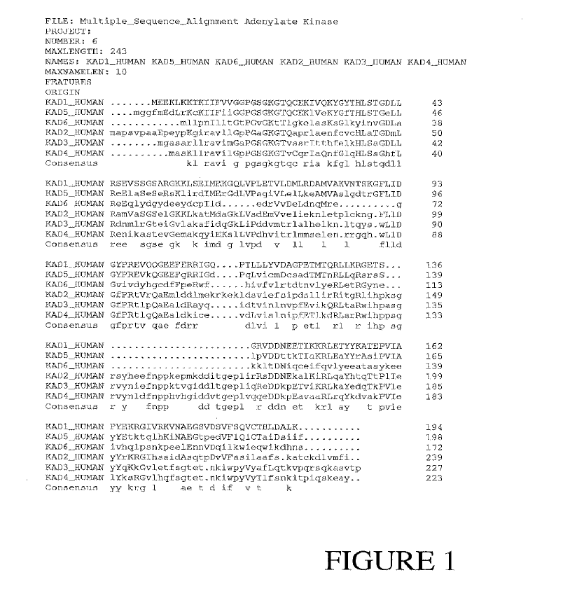

FIG. 1 shows, for Example 1, a protein sequence alignment of human adenylate kinase isoforms 1 to 6. KAD1=P00568 (SEQ ID NO. 1), KAD232 P54819 (SEQ ID NO. 2), KAD3=Q9UIJ7 (SEQ ID NO. 3), KAD4=P27144 (SEQ ID NO. 4), KAD5=Q9Y6K8 (SEQ ID NO. 5), and KAD6=Q9Y3D8 (SEQ ID NO. 6);

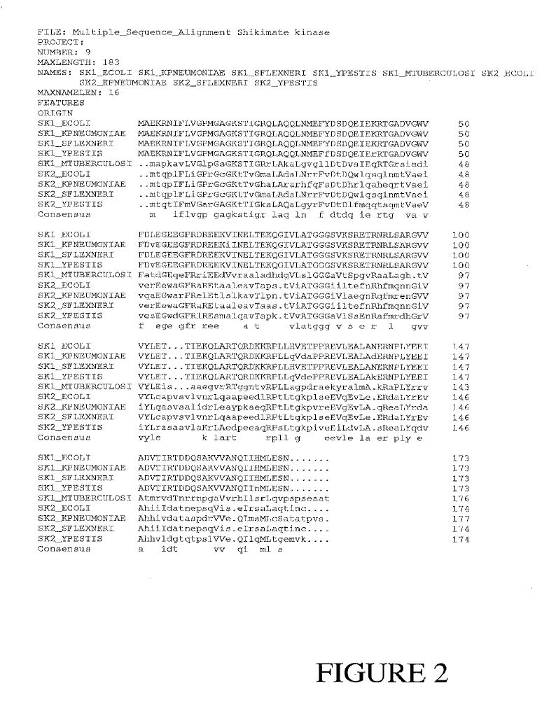

FIG. 2 shows, for Example 1, a protein sequence alignment of shikimate kinase isoforms 1 and 2: E. coli aroK, SK1_{—}Ecoli=P0A6D7 (SEQ ID NO. 7), E. coli aroL SK2_{—}Ecoli=P0A6E1 (SEQ ID NO. 8); Klebsiella pneumoniae aroK, SK1_{—}Kpueumoniae=A6TF14 (SEQ ID NO. 9), Klebsiella pneumoniae aroL, SK2_{—}Kpueumoniae=A6T5B3 (SEQ ID NO. 10); Yersinia pestis aroK, SK1_{—}Ypestis=A6BW25 (SEQ ID NO. 11), Yersinia pestis aroL, SK2ypestis=A4TPJ4 (SEQ ID NO. 12); Shigella flexneri aroK, SK1_{—}Sflexneri=Q0SZS8 (SEQ ID NO. 13), Shigella flexneri aroL, SK2_{—}Sflexneri=Q83M66 (SEQ ID NO. 14), and Mycobacterium tuberculosis aroK, SK1_{—}Mtuberculosis=P0A4Z2 (SEQ ID NO. 15);

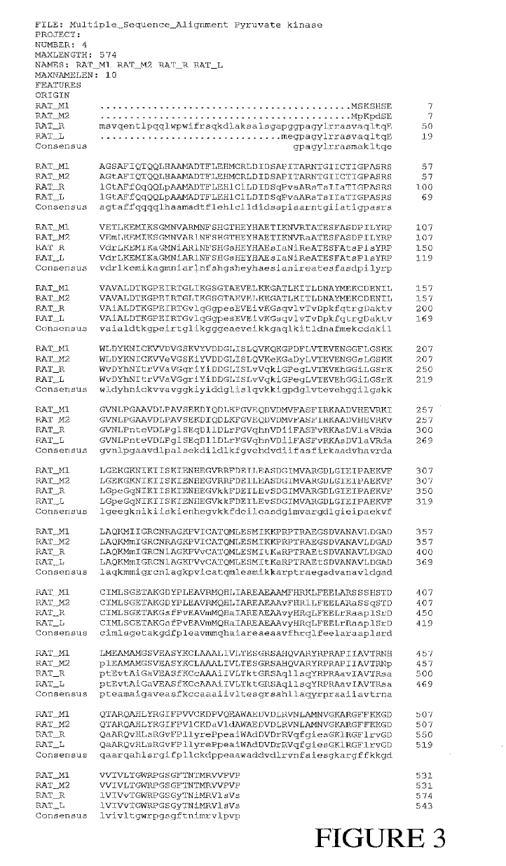

FIG. 3 shows, for Example 1, a protein sequence alignment of pyruvate kinase isoforms R, L, M1 and M2. R=P12928 (SEQ ID NO. 16), L=P04763 (SEQ ID NO. 17), M1=P11980 (SEQ ID NO. 18) and M2=P11981 (SEQ ID NO. 19);

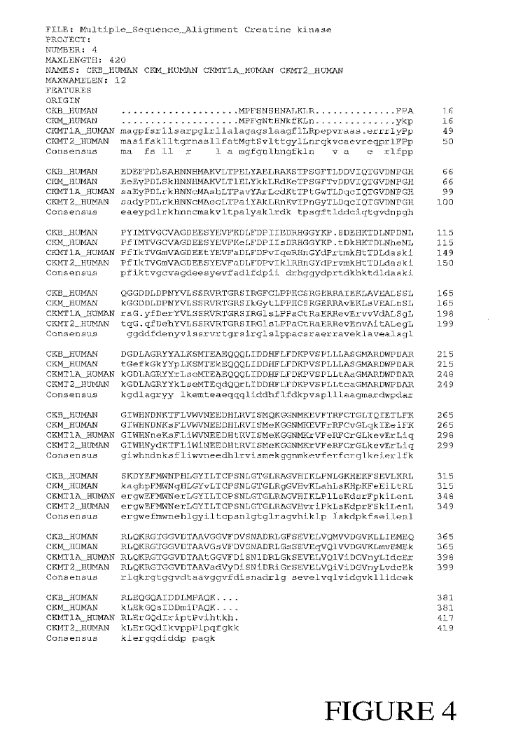

FIG. 4 shows, for Example 1, a protein sequence alignment of creatine kinase isoforms B-CK, cytoplasmic muscle M-CK, uMT and sMT: CKB=P12277 (SEQ ID NO. 20), CKM=P06732 (SEQ ID NO. 21), CKMT1A=P12532 (SEQ ID NO. 22), CKMT2=P17540 (SEQ ID NO. 23);

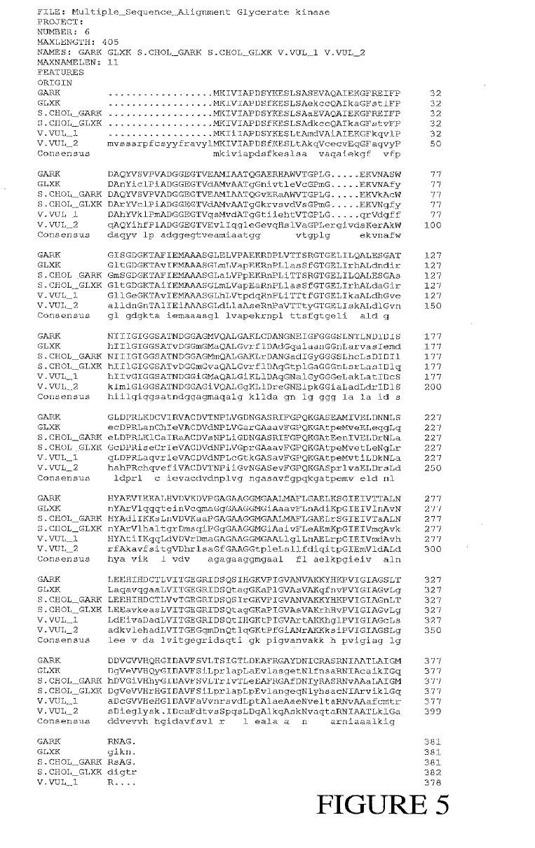

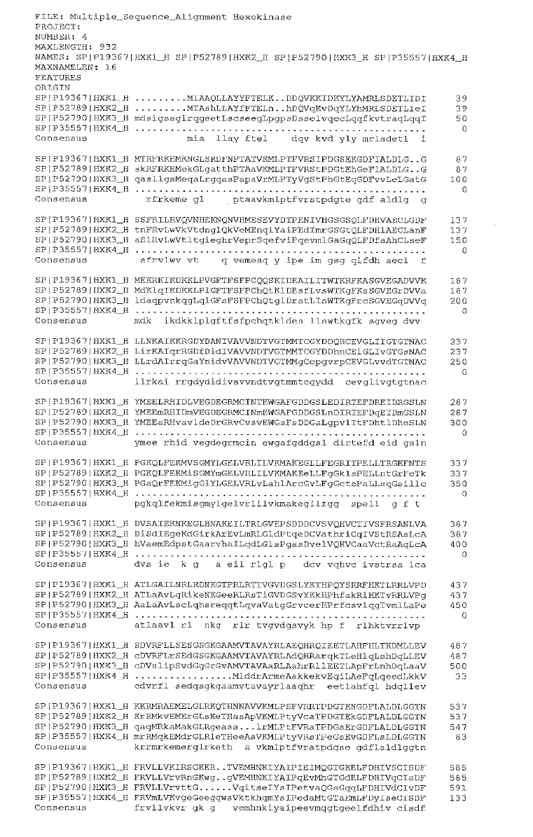

FIG. 5 shows, for Example 1, a protein sequence alignment of glycerate kinase isoforms 1; (SEQ ID NO. 24) and 2 (SEQ ID NO 25).;

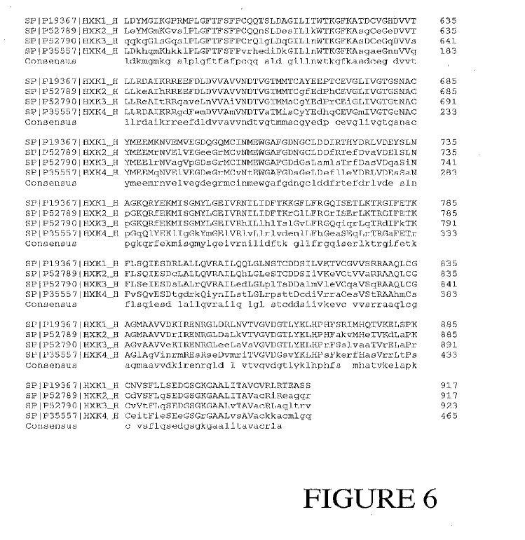

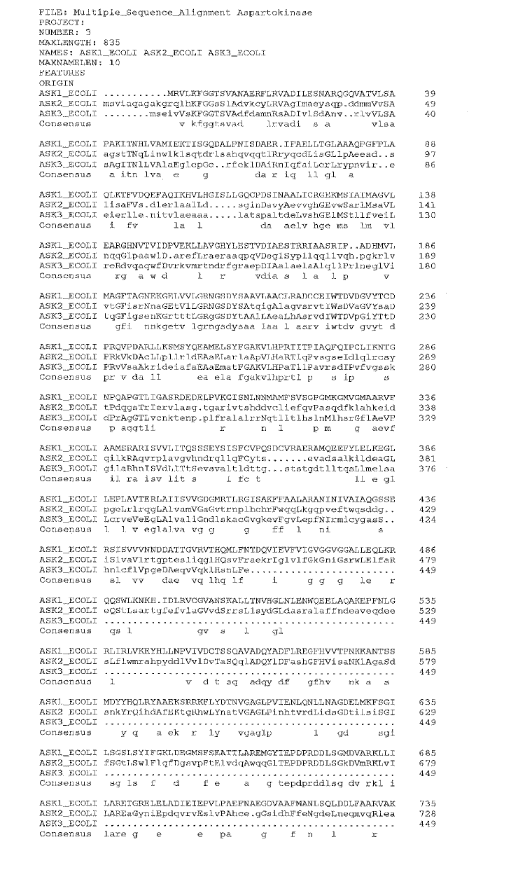

FIG. 6 shows, for Example 1, a protein sequence alignment of human hexokinase isoforms 1-4 (SEQ ID NO. 26 to 29, respectively);

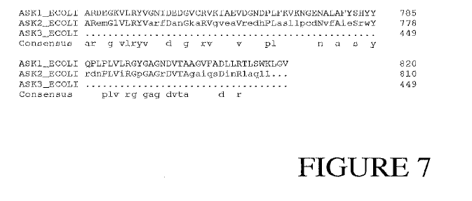

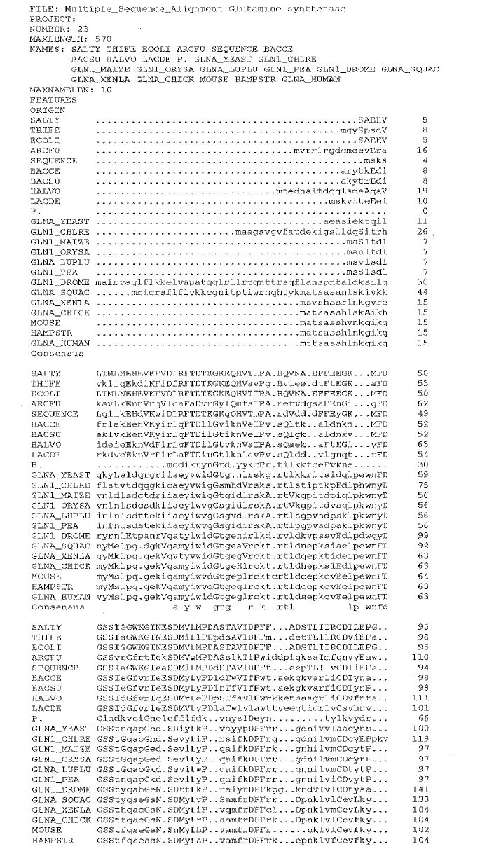

FIG. 7 shows, for Example 1, a protein sequence alignment of E. coli aspartokinase isoforms 1-3 (SEQ ID NO. 30 to 32, respectively);

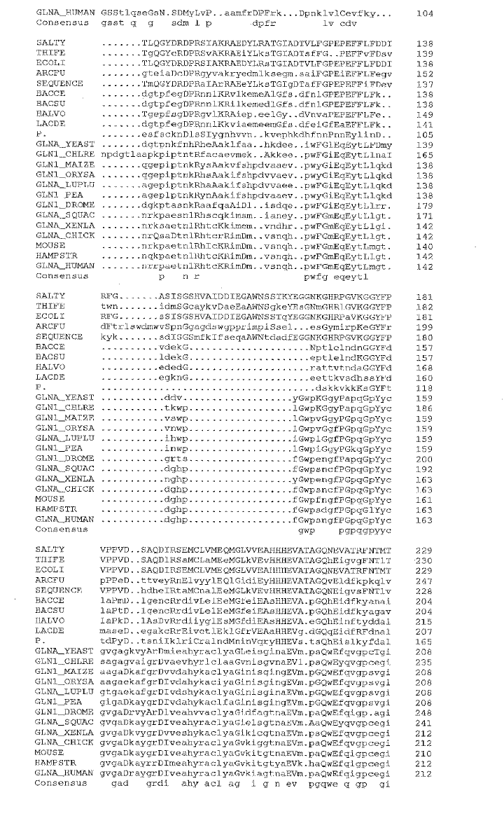

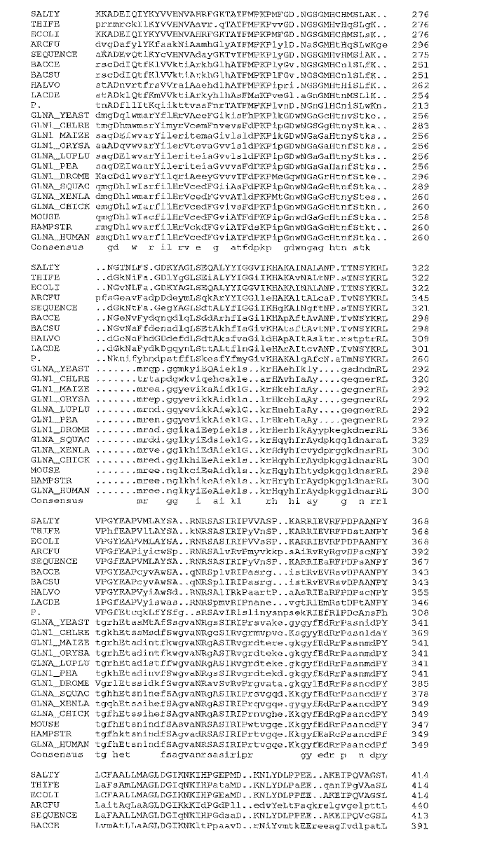

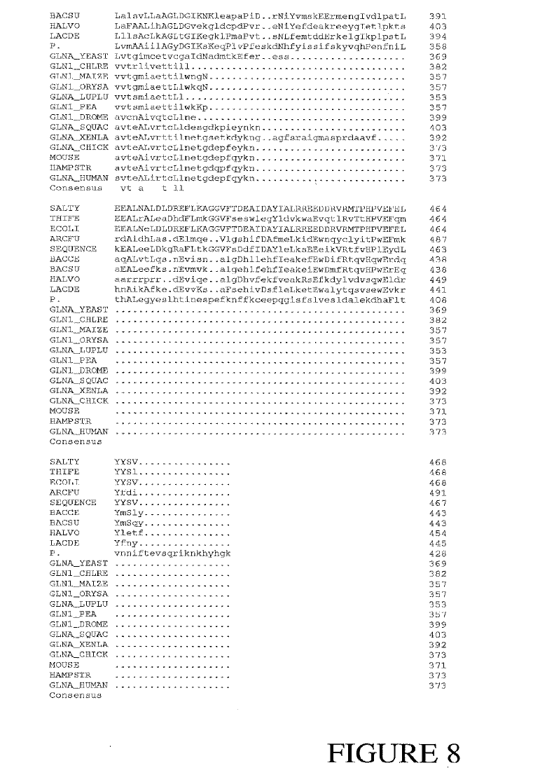

FIG. 8 shows, for Example 1, conserved ATP binding domain sequence motifs in GSI-β compared to the domains in GS1-α and GSII: GLNA_SALTY=Salmonella typhimurium (P0A1 P6, SEQ ID NO 33), GLNA_THIFE=Acidithiobacillus ferrooxidans (P07804, SEQ ID NO 34), GLNA_{—}ECOLI=E. coli (P0A9C5; SEQ ID NO 35), GLNA_ARCFU=Archaeoglobus fulgidus ((O29380; SEQ ID NO 36), GLNA_AZOVI=Azotobacter vinelandii (P22248; SEQ ID NO 37), GLNA_Bacce=Bacillus cereus (P19064; SEQ ID NO 38), GLNA_BACSU=Bacillus subtilis (P12425; SEQ ID NO 39), GLNA_HALVO=Halobacterium volcanii (P43386; SEQ ID NO 40), GLNA_LACDE=Lactobacillus delbrueckii (P45627; SEQ ID NO 41)GLNA_PLASMO=Plasmodium falciparum (NCBI: XP001352097; SEQ ID NO 42), GLNA_YEAST=Saccharomyces cerevisiae (P32288; SEQ ID NO 43), GLNA_CHLRE−Chlamydomonas reinhardtii (Q42688; SEQ ID NO 44), GLNA_MAIZE−Zea mays (P49094; SEQ ID NO 45), GLNA_ORYSA=Orysa sativa (P14656; SEQ ID NO 46), GLNA_LUPLU−Lupinus luteus (P52782; SEQ ID NO 47), GLNA_PEA=Pisum sativum (P19251; SEQ ID NO 48), GLNA_DROME−Drosophila melanogaster (P20477; SEQ ID NO 49), GLNA_SQUAC=Squalus acanthia (P41520; SEQ ID NO 50), GLNA_XENLA =Xenopus laevis (P51121; SEQ ID NO 51), GLNA_CHICK=Gallus gallus (P16580; SEQ ID NO 52), GLNA_MOUSE=Mus musculus (P15105; SEQ ID NO 53), HAMSTR=Cricetulus griseus (PO4773; SEQ ID NO 54) and GLNA_HUMAN=Homo sapiens (P15104; SEQ ID NO 55).

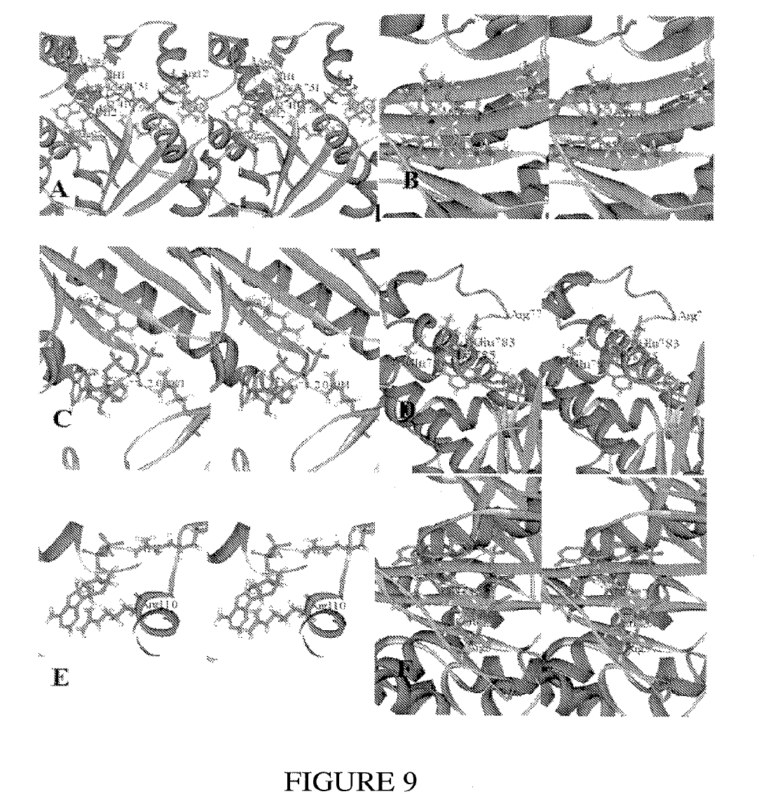

FIG. 9 shows, for Example 1, stereo views of the interaction of identified Arg and Glu/Asp interaction in the active sites of in a range of kinase isoenzymes and a synthetase enzyme, namely AK (A), CK (B), PK (C), HXK, SK (D), ASK (E) and GS (F);

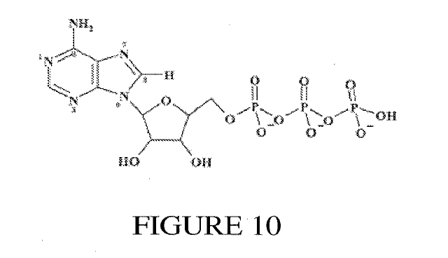

FIG. 10 shows, for Example 1, structure and numbering of ATP;

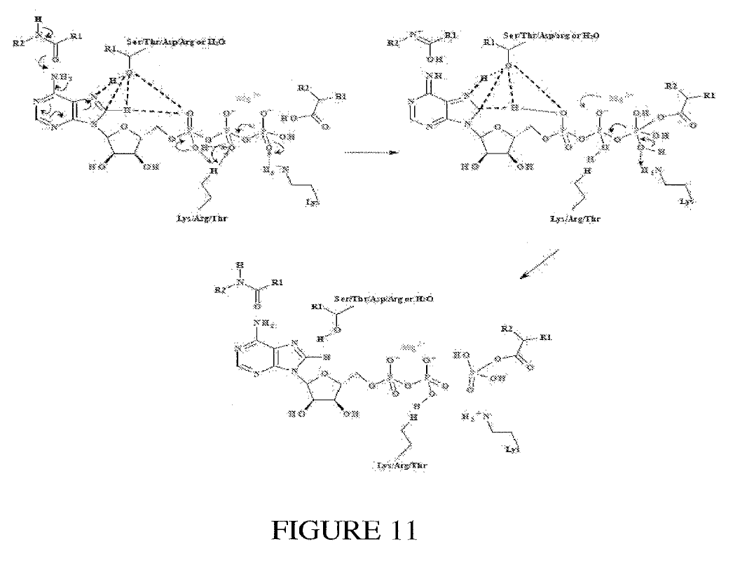

FIG. 11 shows, for Example 1, the role of Arg97 and Arg44 and the C8H of ATP in the binding of ATP and catalysing phosphoryl transfer in ATP dependent reactions;

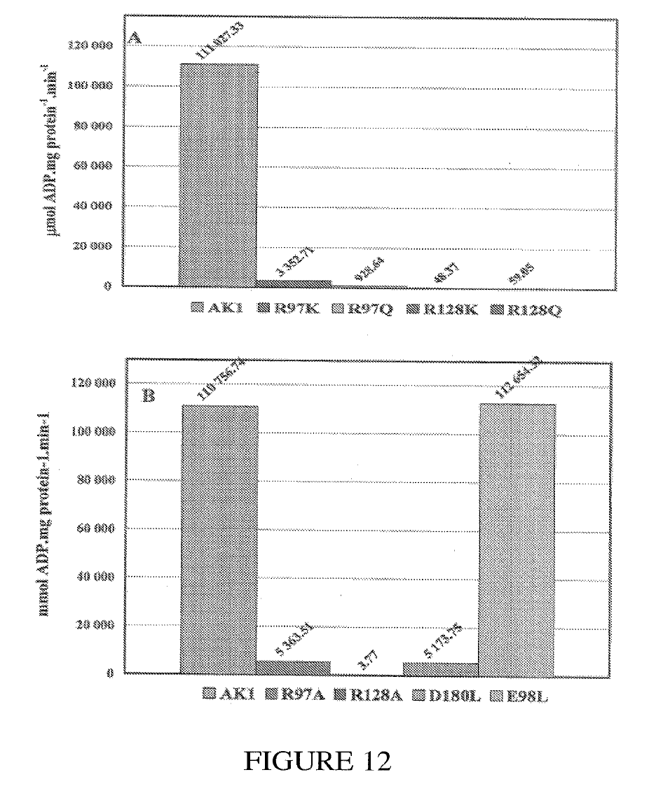

FIG. 12 shows, for Example 2, the effect of the SDM R97K, R97Q, R97A, R128K, R128Q, R128A, E98L and D180L on the specific activity of AK1 with (A): R97K, R97Q, R128K, R128Q, and (B): R97A, R128A E98L and D180L;

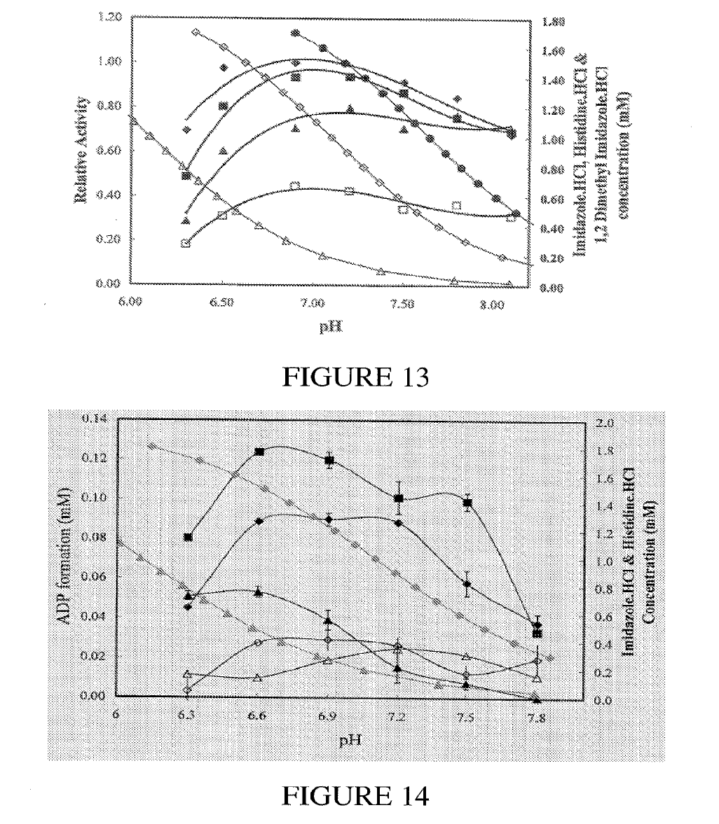

FIG. 13 shows, for Example 3, the effect of pH and NaCl (♦), imidazole (▪), histidine (▴) and 1,2 dimethyl imidazole (□) on the enzyme activity of AK1. Activity is expressed as ADP produced in mM. Effect of pH on the protonation of imidazole (⋄), histidine (Δ) and 1,2 dimethyl imidazole ();

FIG. 14 shows, for Example 4, the effect of imidazole.HCl and histidine.HCl and deuterated imidazole.HCl and histidine.HCl at a range of pH values on the activity of AK1, (▪) 2 mM NaCl, (♦) imidazole.HCl, (▴) histidine.HCl, (⋄) deuterated imidazole.HCl and (□) deuterated histidine.HCl;

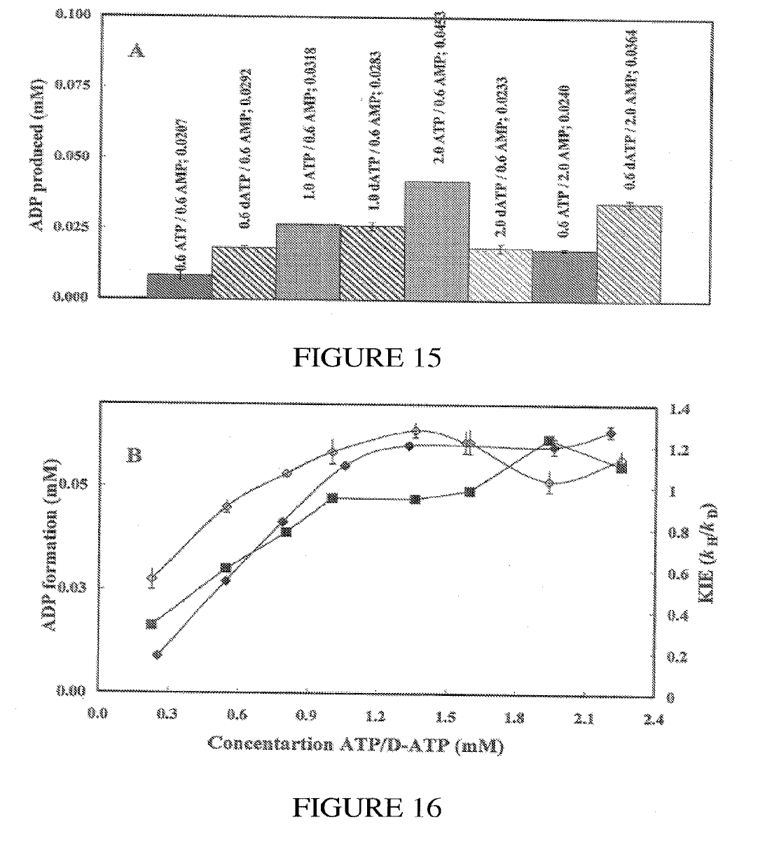

FIG. 15 shows, for Example 5, the effect of the relative concentrations of ATP, AMP and C8-D ATP on the activity of AK1;

FIG. 16 shows, for Example 5, the effect of a range of concentrations of ATP (♦) and C8-D ATP (⋄) on the activity of AK1 and the KIE (▪);

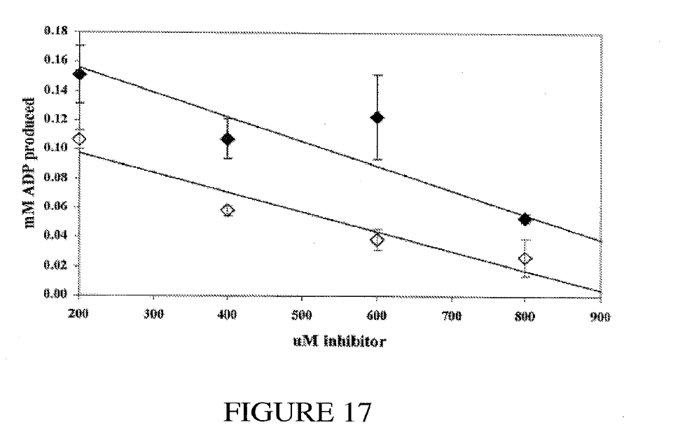

FIG. 17 shows, for Example 6, the effect of Adenosine 5′-[y-thio]triphosphate (ATPS) (♦) and deuterated ATPS (⋄) on the synthesis of ADP by AK1. The activity of AK1 in the absence of ATPS produced 0.019 mM ADP; and

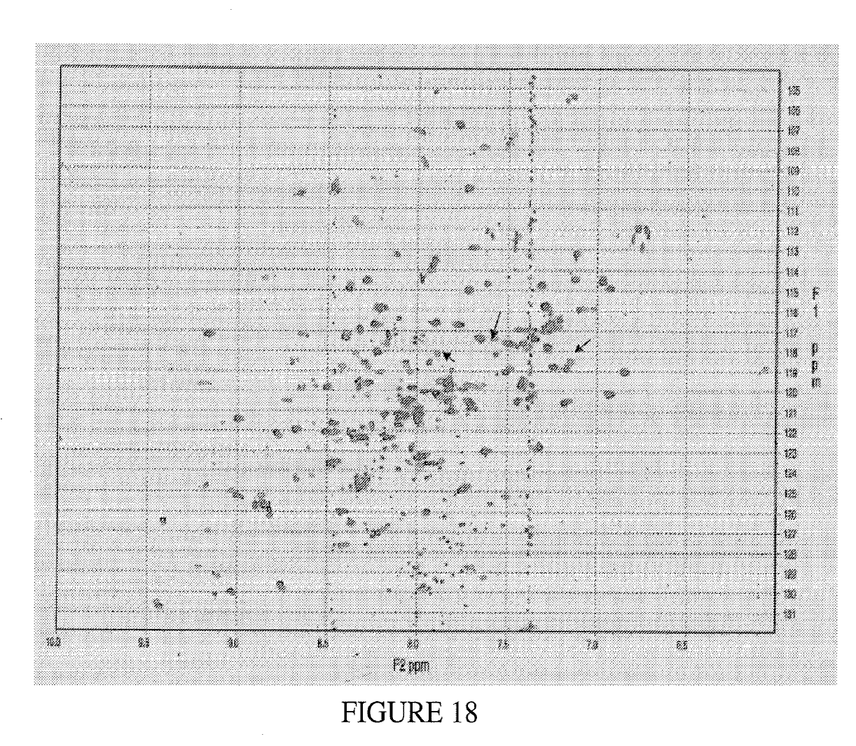

FIG. 18 shows, for Example 7, ¹H and ¹⁵N NMR HSQC spectrum of AK1 in the presence of ¹H-ATPS (light shading) and C8-D ATPS (darker shading). Arrows indicate shift changes as a result of the binding of the deuterated analogue.

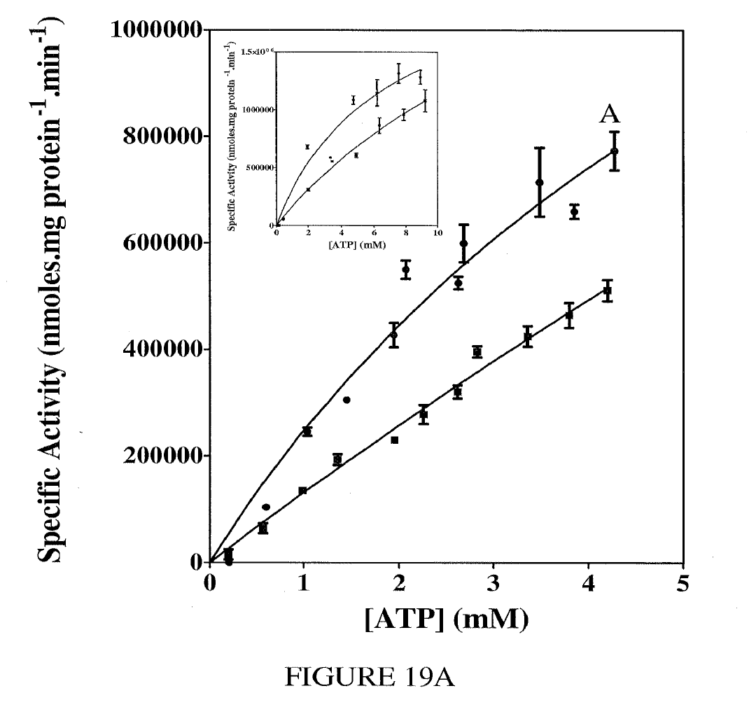

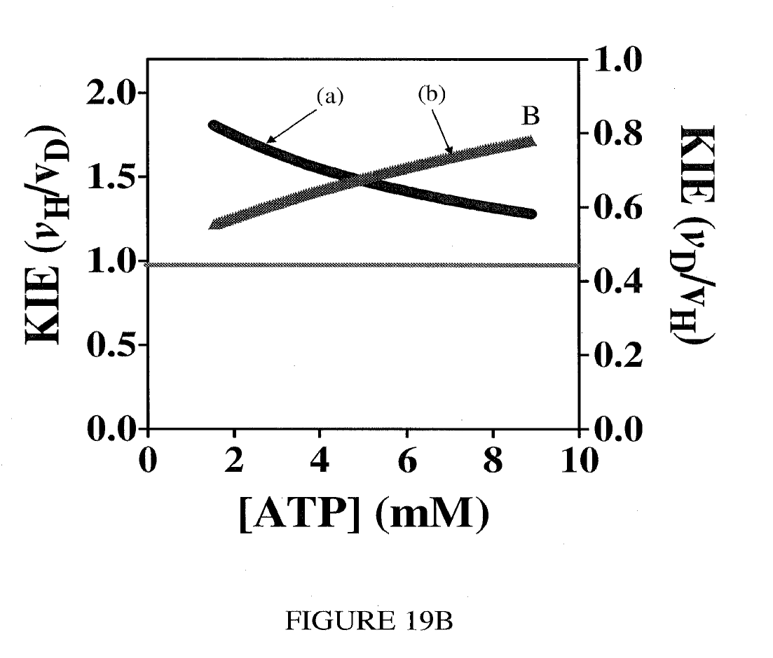

FIG. 19 shows, for Example 8, the effect of the concentration of ATP and C8D-ATP on the specific activity (A) and KIE (B) of Mycobacteria tuberculosis shikimate kinase; =ATP, ▪=C8D-ATP, a=KIE, b=KIE_{D}.

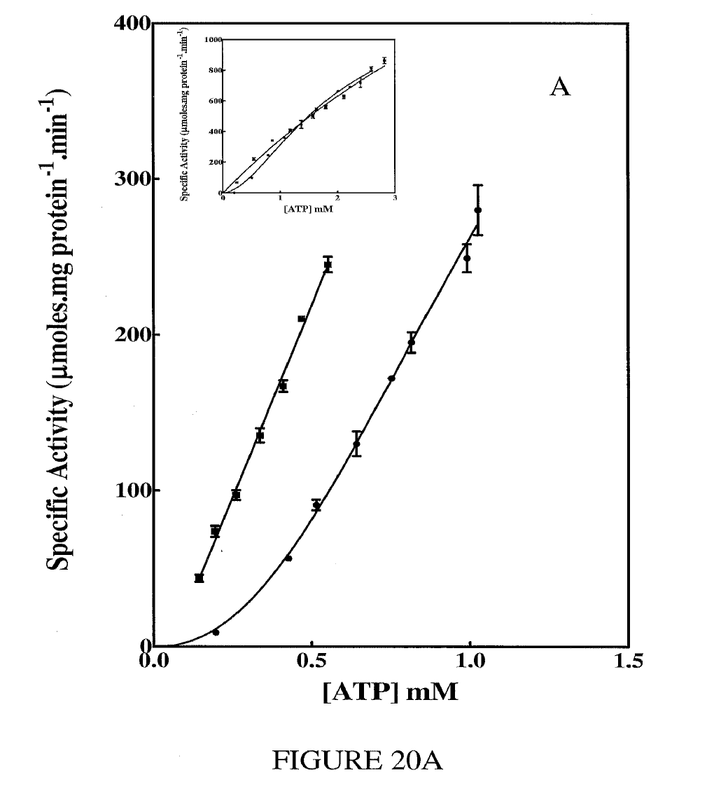

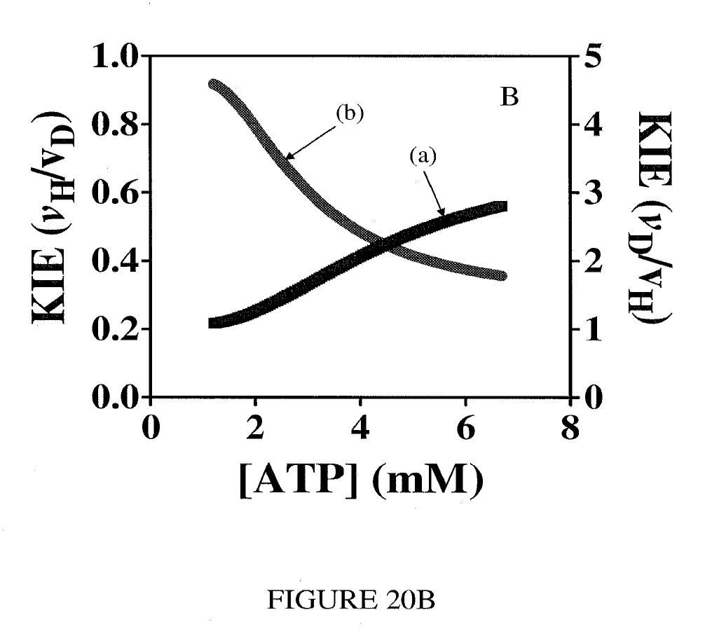

FIG. 20 shows, for Example 8, the effect of the concentration of ATP and C8D-ATP on the specific activity (A) and KIE (B) of Saccharomyces cerevisiae hexokinase; =ATP, ▪=C8D-ATP a=KIE, b=KIE_{D}.

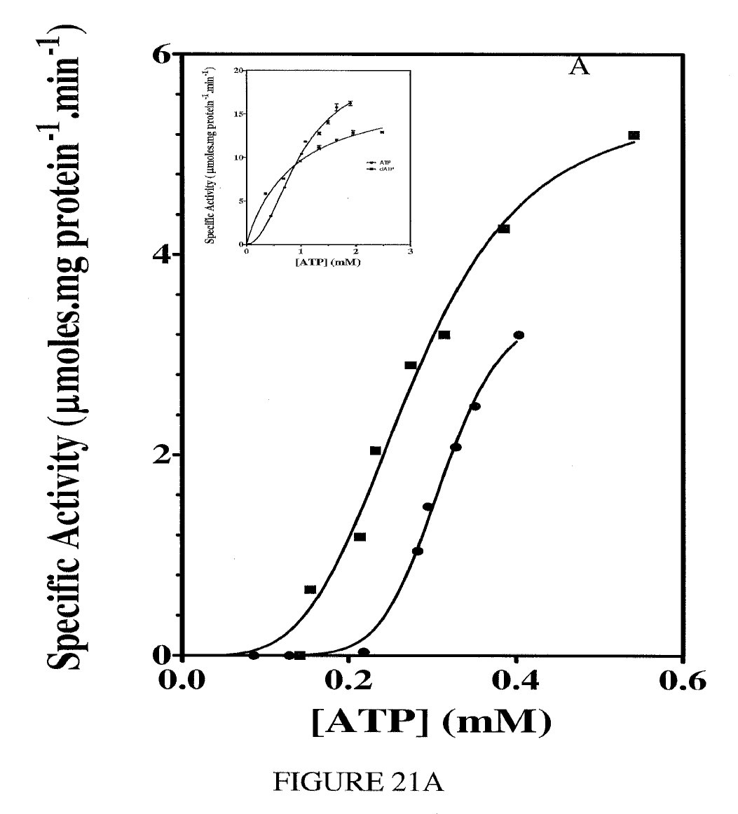

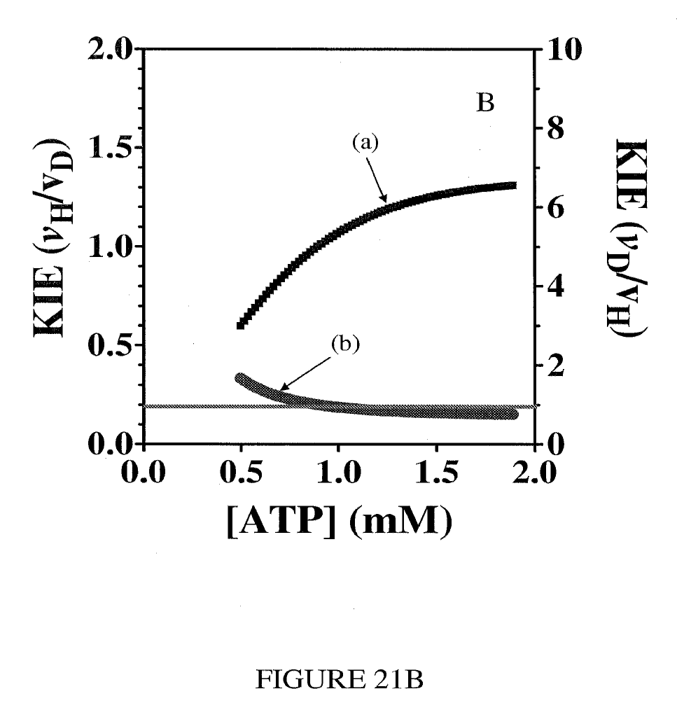

FIG. 21 shows, for Example 8, the effect of the concentration of ATP and C8D-ATP on the specific activity (A) and KIE (B) of Escherichia coli acetate kinase; =ATP, ▪=C8D-ATP, a=KIE, b=KIE_{D}.

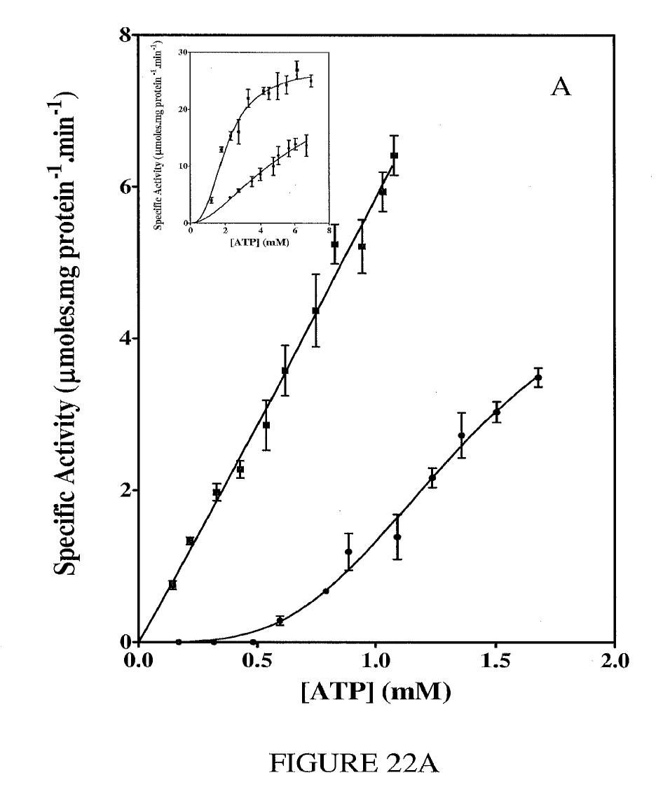

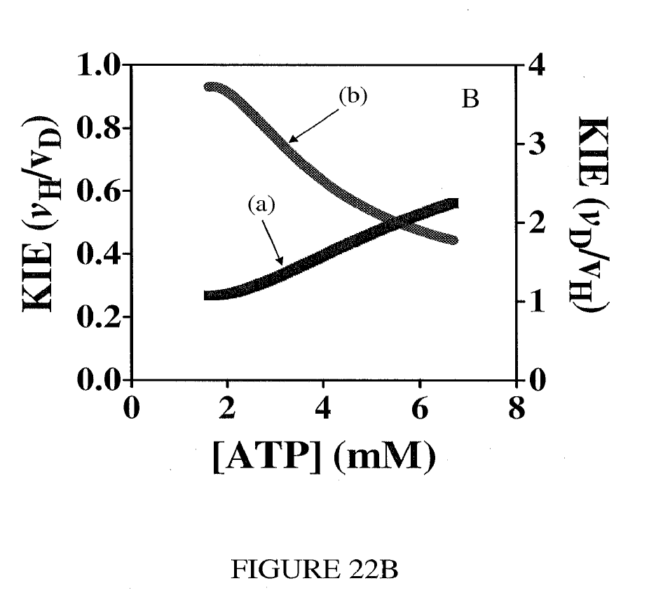

FIG. 22 shows, for Example 8, the effect of the concentration of ATP and C8D-ATP on the specific activity (A) and KIE (B) of Escherichia coli GS₀; =ATP, ▪=C8D-ATP a=KIE, b=KIE_{D}.

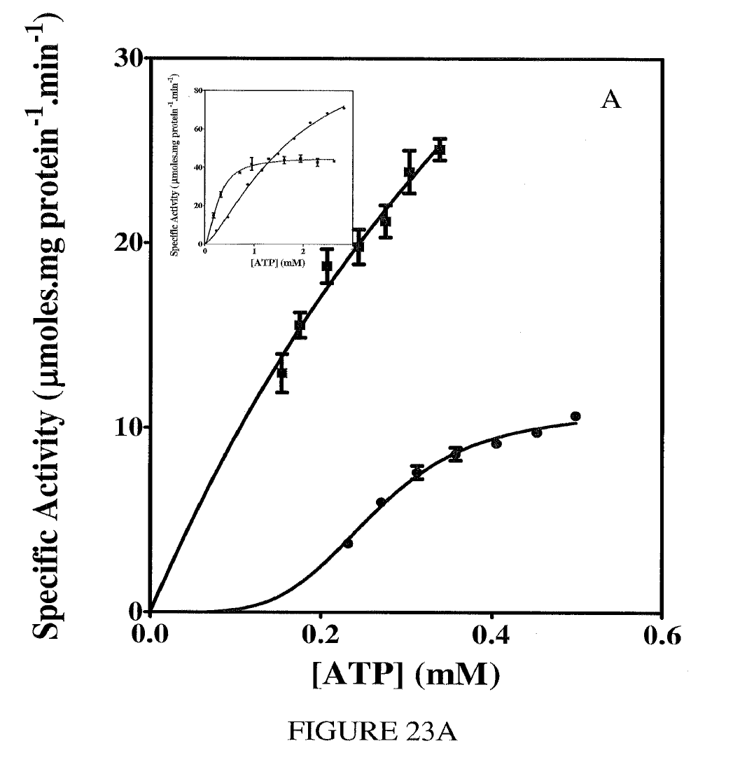

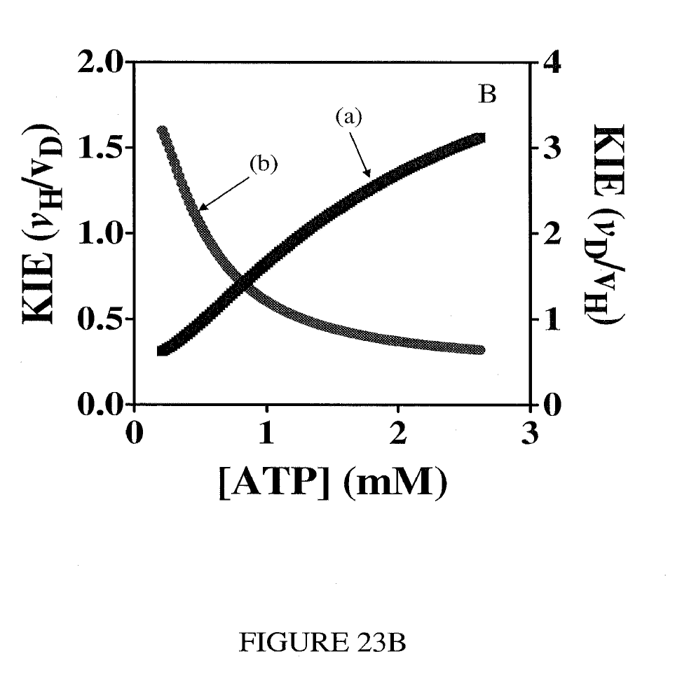

FIG. 23 shows, for Example 8, the effect of the concentration of ATP and C8D-ATP on the specific activity (A) and KIE (B) of B. stearothermophilus phosphofructokinase; =ATP, ▪=C8D-ATP, a=KIE, b=KIE_{D}.

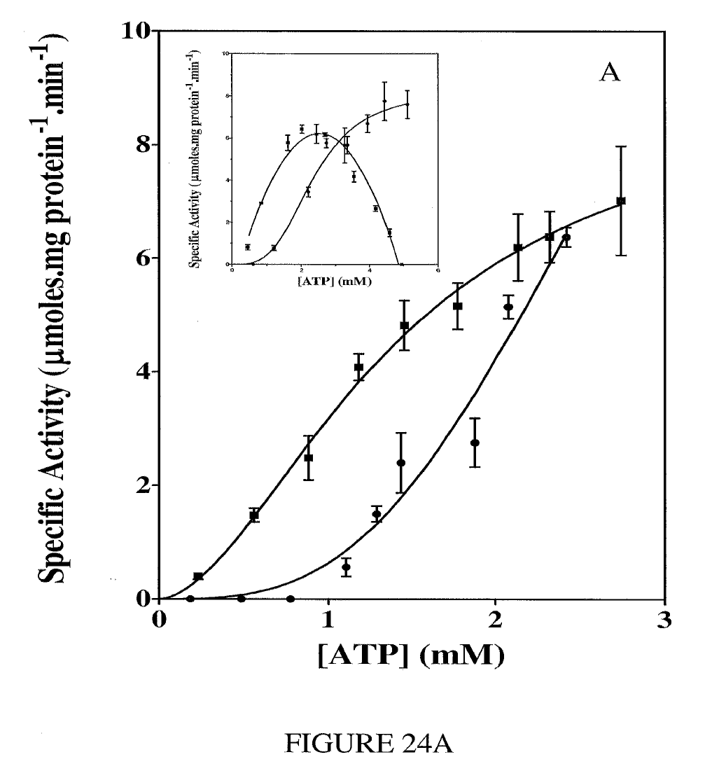

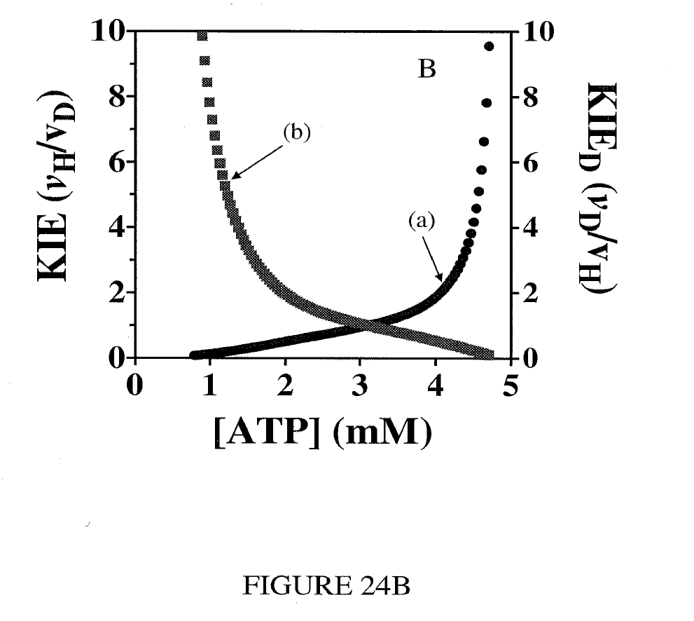

FIG. 24 shows, for Example 8, the effect of the concentration of ATP and C8D-ATP on the specific activity (A) and KIE (B) of Escherichia coli adenylylated glutamine synthetase; =ATP, ▪=C8D-ATP, a=KIE, b=KIE_{D}.

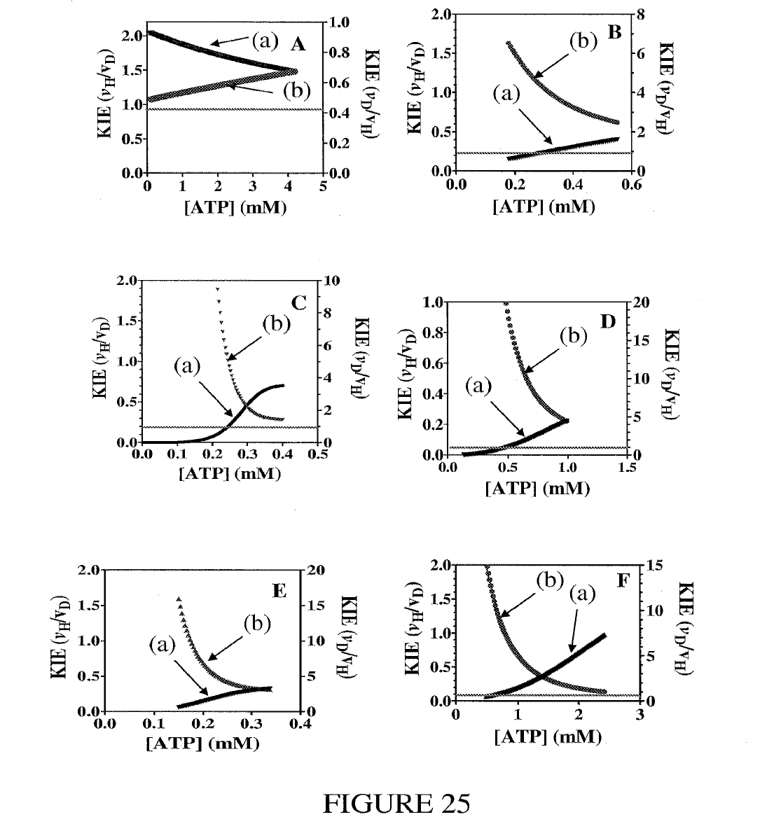

FIG. 25 shows, for Example 8, the effect of low concentrations of ATP on the KIE and KIE_{D} obtained for A: shikimate kinase, B: hexokinase, C: acetate kinase, D: GS₀, E: PFK and F: GS₁₂; a=KIE, b=KIE_{D}

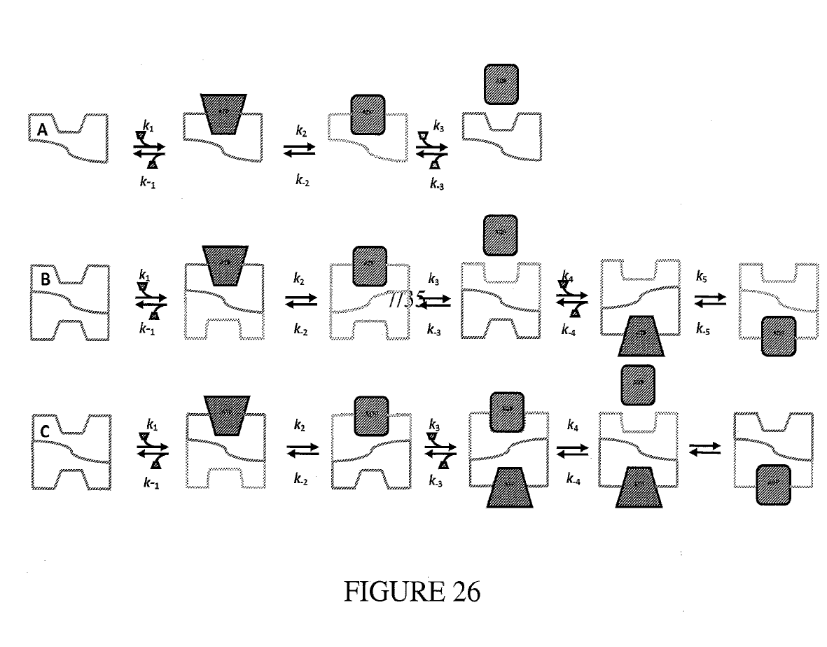

FIG. 26 shows, for Example 8, models for the binding of nucleotides to kinases and synthetase enzymes. A: Model for the binding of ATP and release of ADP from monomeric kinase. The current model for oligomeric kinases is based on nonequivalent ligand binding where binding to one monomer affects binding to a second monomer, or coordinated active sites. B: Model based on the rate reaction in the conversion of ATP to ADP with the concomitant conversion of the second binding site to the ATP-binding-form as a result of the release of the ADP. Once the ADP has been released from the first site this changes the affinity of the second site from an ADP binding structure to an ATP binding structure. C: Model based on the rate reaction in the conversion of ATP to ADP with the concomitant conversion of the second binding site to the ATP-binding-form as a result of the conversion of ATP to ADP in the first binding site. Once the ATP is converted ADP this changes the affinity of the second site from an ADP-binding structure to an ATP-binding-form.

### Example 1

The kinases are a large number of structurally diverse enzymes that play a critical role in numerous metabolic and signaling pathways and whose substrates may be a small molecule, lipid, or protein. The kinases have been classified into 25 families of homogenous proteins, with the families assembled into 12 fold-groups based on the similarity of their structural folds. The enzymes transferring high energy phosphate bonds from nucleotides into two divisions, namely, transferases (kinases) and ligases (synthetases). The catalytic and regulatory mechanisms employed in nucleotide binding and phosphoryl transfer, within a single kinase group from both prokaryotic and eukaryotic organisms, as well as the specific kinase isoenzymes, are kinetically and functionally distinct based on the rate of phosphoryl transfer and the regulation thereof. An initial structural comparison of key amino acid residues associated with phosphoryl transfer activity and the regulation of kinase enzymes and their isoenzymes, as depicted in FIGS. 1 to 8 and in Table 1, demonstrated the level of structural homology of the residues that are associated with phosphoryl transfer activity and/or the regulation of phosphoryl transfer activity, and whether functionality may be differentiated based on the conservation of key amino acids in the active sites of these kinases and the role of the conserved amino acid residues in phosphoryl transfer. The sequence and structural analysis of a range of kinase and synthetase enzymes was carried out using the Accelrys INSIGHTII and Discovery Studio 2.5 suite of molecular modeling software—see FIGS. 1 to 9. The protein sequence alignments of each enzymes isoforms were carried using the DNAMAN sequence alignment software using the dynamic alignment with a gap penalty of 10, a gap extension penalty of 5 and the BLOSUM protein weight.

Based on the structural analysis of the nucleotide binding site of a range of diverse kinase enzymes, as depicted in FIG. 9, the role of key amino acids involved in nucleotide binding was defined. The protein sequence alignments for each enzyme isoform and structural investigation of the active sites of adenylate kinase (AK), shikimate kinase (SK), pyruvate kinase (PK), hexokinase (HXK), aspartokinase (ASK) and creatine kinase (CK) indicated conserved paired Arg and Glu/Asp in the active site associated with the binding of the imidazole moiety and the α-PO₄ of the nucleotide (Table 1 and FIG. 9) (Sequence alignments: FIGS. 1-8, Structural comparison of conserved residues: FIG. 9 and Table 1). The AK group of enzymes isoforms served as a template for the identification of key amino acid residues that could be required for the binding and/or regulation of phosphoryl transfer within the active site of kinases. The mechanism of binding and the role of the imidazole moiety of the nucleotide in the binding and its role in the regulation of catalysis was defined. Within the active site of AK, SK, PK, HXK, ASK and CK, Arg and Glu/Asp residues associated with the binding ATP to the active site of the enzymes (Table 1), are conserved. The NH2 of the Arg97 is within a requisite 3.5 Å of the C-8 proton (ATP atom numbering: FIG. 10) of the ATP analogue. As the active sites of AK1 and AK4 were not distorted in the crystal structure, these sites were used to identify the imidazole binding domain within the active site. AK1 contains “diadenyl tetraphosphate” in the active site while AK4 contains “diadenyl pentaphosphate” in the active site. Hydrogen bonding occurs between conserved Arg and Glu or Asp acid residues and the imidazole moiety of the nucleotide. AK1 has the Arg 97 and Glu 98 and the pair Arg 128 and Asp 180 associated with the equivalent N7/C8 atoms of the diadenyl tetraphosphate. The equivalent residues in AK2-AK6 are outlined in Table 1. In CK, the Arg/Glu pair hydrogen bonded to the imidazole moiety of the nucleotide ATP are Arg 292 and Asp 335. Outlined in Table 1 and FIG. 9 are similar interactions between the C8-H of the imidazole moiety of the nucleotide and the conserved Arg residue within the active sites of AK, SK, PK, HXK, ASK, CK and GS isoenzymes. In all enzymes investigated, other than HXK I-III, a conserved Arg was identified within approximately 3.5 A of the C8-H of the imidazole moiety of the associated nucleotide. In HXK I-III, the Arg is replaced by a Lys whereas in HXK IV the Arg is conserved. The identified Arg residue was also found to be H-bonding distance of the α-PO₄ of the nucleotide. It is proposed the concomitant association of the Arg with the C8-H of the imidazole moiety and the α-PO₄ of the nucleotide has mechanistic implications in the regulation of the phosphoryl transfer activity of the enzyme. In all cases additional arginine residue/s are associated with the primary arginine residue which is associated with the imidazole moiety of the nucleotide. The second arginine residue is also associated with the phosphate backbone of the nucleotide. The arginine residues may be replaced by either glutamine or lysine.

It was therefore proposed that the C-8 proton of ATP plays a direct role in the binding of ATP to the active site as well as in the catalysis of phosphorylation by general acid-base catalysis as outlined in FIG. 11. The role of the Arg97 is to act as a general base catalyst in the abstraction of the C-8 proton from ATP which in turn, via resonance stabilization, acts to protonate the α-phosphate of the ATP. The second arginine in the active site, Arg 44, then acts to transfer the proton to the β-phosphate. This acid-catalysed stabilization of the α- and β-phosphates of the ATP acts to withdraw electrons into the “backbone” of the, α-β-γ-phosphate chain of ATP, allowing for resonance stabilization of the phosphate backbone and facilitating that the γ-phosphate acts as a better leaving group in the phosphorylation of the substrate molecules.

TABLE 1
Identified Arg and Glu/Asp residues responsible for the regulation of phosphoryl transfer in a range of kinase isoenzymes and a synthetase enzyme.
The rows contain the conserved amino acid residues from various isoenzymes of each enzyme. Where structures contained a co-crystallized
nucleotide the inter-atomic distances between the C8-H of the imidazole moiety, α-PO₄ of the nucleotide and the amino acid were measured
(Atoms and inter-atomic distances outlined below each residue). Within each isoenzyme the Glu/Asp residues associated with each Arg residue
follow the Arg residue in the column of the table. In PK, AK and CK the Arg is also in H-bonding distance of the C8-H of ATP as well the α-PO₄
of ATP. In SK1 a second Arg residue is responsible for the interaction with C8-H of ATP as well the α-PO₄ of ATP.

| | | PK- | AK1^{a} | AK5 | AK6 | AK2 | AK3 | AK4 | M-CK | uMt-CK | sMt-CK |
|---|---|---|---|---|---|---|---|---|---|---|---|
| PK-R | PK-L | M1/M2^{a} | (Cyto) | (Cyto) | (Cyto) | (Mito) | (Mito) | (Mito) | (Cyto) | (Mito) | (Mito)^{a} |
| His121 | His89 | His77 | Arg97 | Arg100 | Arg? | Arg103 | Arg94 | Arg92 | Arg292 | Arg325 | Arg326 |
| His ND₁ to ATP-C8H = 4.57 Å | | | Arg NH₁ to ATP-α-PO₄ = 2.41 Å | | | | | | Arg NH₂ to ATP-C8H = 2.58 Å | | |
| His NE2 to ATP-α-PO₄ = 1.92 Å | | | Arg NH₂ to ATP-C8H = 3.46 Å | | | | | | Arg NH₂ to ATP-α-PO₄ = 3.58 Å | | |
| His NE2 to Arg120 NH1 = 1.92 Å | | | | | | | | | Arg HE to ATP-β-PO₄ = 1.67 Å | | |
| Arg116 | Arg84 | Arg72 | Glu98 | Glu101 | Asp77? | Glu109 | Glu100 | Glu98? | Arg236 | Arg269 | Arg270 |

| Arg NH₁ to ATP-C8H = 4.60 Å | | | | | | | Arg NH₁ to ATP-α-PO₄ = 3.15 Å | | |
|---|---|---|---|---|---|---|---|---|---|
| Arg NH₁ to ATP-α-PO₄ = 1.92 Å | Arg44 | Arg47 | Arg39 | Arg51 | Arg43 | Arg41 | Arg320 | Arg352 | Arg354 |

| Arg NH₁ to ATP-α-PO₄ = 1.90 Å | Arg NH₁ to ATP-α-PO₄ = 1.92 Å | | |
|---|---|---|---|
| Arg NH₁ to ATP-α-PO₄ = 3.75 Å | Asp335 | Asp368 | Asp369 |

| | | | | | | | | | |
|---|---|---|---|---|---|---|---|---|---|
| Arg163 | Arg132 | Arg120 | Arg128 | Arg131 | Arg105 | Arg138 | Arg124 | Arg122 | Asp δ-OD₁ to Arg326 NH₁ = 1.85 Å |

| Arg NH₁ to ATP-β-PO₄ = 2.05 Å | | Arg NH₁ to ATP-C8H = 6.40 Å | | | | | | |
|---|---|---|---|---|---|---|---|---|
| | | Asp180 | Asp184 | Asp157 | Glu? | Glu200 | Glu198 | |
| SK1^{a,b} | SK2^{b} | HK1/HK2^{a} | HK3 | HK4 | ASK1 | ASK2 | ASK3 | GS |
| Arg113 | Arg NE | Lys785^{c} | Lys791 | Arg333 | Arg238 | Arg241 | Arg232 | Arg 355 |

| | | |
|---|---|---|
| Arg HE to Glu108 δ-OD₁ = 2.39 Å | Arg NH₁ to ATP-C8H = 4.33 Å | Arg NH₂ to ATP-C8H = 3.95 Å |
| Arg NH₁ to ATP-C8H = 4.00 Å | Arg NH₂ to His271 ND1 = 3.33 Å | |

| | | | | | | | |
|---|---|---|---|---|---|---|---|
| Glu108 | Glu NE | Gln789 | Gln NE | Gln337 | Arg306 | Arg309 | Arg300 |

| | | | | | |
|---|---|---|---|---|---|
| Arg120 | Arg117 | Arg30^{d} | Lys41 | NE | Glu 357 |

| | | | | |
|---|---|---|---|---|
| Arg NH₁ to ATP-α-PO₄ = 2.75 Å | Arg NH₂ to ATP-C8H = 3.44 Å | Asp228 | Asp231 | Asp222 |

| | |
|---|---|
| Glu? | NE |

Asp OD₁ to Arg30 NH₁ = 3.25 Å

The PDB accession codes for protein structures used, pyruvate kinase (PK); 1A49, adenylate kinase (AK1); 2C95, creatine kinase (CK); 2GL6, Shikimate kinase (SK1), 1L4U, hexokinase (HXK1) catalytic site; 1DGK, hexokinase (HXK1) allosteric regulation site; 1QHA, glutamine synthetase (GS): 1F52.
^{a}Isoenzyme from which the associated inter-atomic distance data was obtained.
^{b}Residue number taken from E. coli sequence however Arg/Glu/Asp identification in structure taken from Mycobacteria tuberculosis structure as ADP is co-crystallized into the active site of this structure.
^{c}Catalytic subunit active site residue.
^{d}Allosteric regulation subunit binding site residue.
NE = No equivalent residue in this isoenzyme

### Example 2: Determination of the Role of Key Amino in AK1 Using Site-Directed Mutagenesis

The role of key amino acids involved in the nucleotide binding was demonstrated by site-directed mutagenesis of AK1 and enzymatic analysis of the mutated enzymes demonstrating the necessity of these amino acids in enzyme activity and nucleotide binding.

Construct AK1A-c001, encoding a N-terminal His-tagged human adenylate kinase 1 gene, was obtained from the Structural Genomics Consortium, University of Oxford, United Kingdom. Site-directed mutagenesis was carried out using Finnzymes' Phusion Site-Directed Mutagenesis kit. The mutations created were: Arg97 and Arg128 to Ala, Gln and Lys, and Glu98 and Asp180 to Leu. All resulting constructs were sequenced to confirm mutations. Wild-type and mutated versions of His-tagged AK1 were expressed in E. coli Origami(DE3) (Novagen), and purified using Bio-Rad's Profinia Purification System. Purified proteins were dialysed against 50 mM KH₂PO₄/K₂HPO₄ buffer (pH 6.8) containing 1.5 mM MgCl₂ and 120 mM KCl.

The effect of the SDM on the specific activity of AK1 was determined in assays containing 50 mM KH₂PO₄/K₂HPO₄, 0.6 mM ADP, 0.6 mM ATP and 0.66 mM MgCl₂ at 37° C. at pH 6.9. The reaction was stopped by the addition of trichloroacetic acid to a final pH of 2 to 3. The assay solutions were centrifuged prior to HPLC analysis. The assays for adenosine, AMP, ADP and ATP were carried out using a Phenomenex 5 μ LUNA C₁₈ column with the mobile phase containing PIC A® (Waters Corporation), 250 ml acetonitrile and 0.7% (w/v) KH₂PO₄. The flow rate of the mobile phase was 1 ml/min with UV detection.

The effect of the following SDM; R97K, R97Q, R97A, R128K, R128Q, R128A, E98L and D180L on the specific activity of AK1 was determined—see FIGS. 12A and B. In the case of both Arg97 and Arg128 mutations to Lys or Gln gave a significant decrease in the specific activity of the enzyme with the effect being 32 fold for R97K, 120 fold for R97Q, and 2200 fold in the case of R128K and R128Q. The R97A mutation gave a 100 fold reduction in activity while the R128A enzyme did not give detectable activity. The D180L mutation gave a 20 fold reduction in activity while the E98L mutation had no effect on the specific activity. The effect of the mutations on the Arg128/D180 pair was greater than the Arg97/Glu98 pair.

### Example 3: Effect of pH on the Inhibition of AK1 by Imidazole.HCl, Histidine.HCl and 1,2 Dimethyl Imidazole.HCl

The effect of the enzyme assay pH and the presence of imidazole.HCl, histidine.HCl and 1,2 dimethyl imidazole.HCl on the activity of AK1 was determined. The assays contained 50 mM KH₂PO₄/K₂HPO₄ (at the equivalent pH), 0.6 mM ADP, 0.6 mM ATP, 0.66 mM MgCl₂ and either imidazole, histidine. or 1,2 dimethyl imidazole at a concentration of 2 mM.

The effect of imidazole.HCl, histidine.HCl and 1,2 dimethyl imidazole.HCl concentration on the activity of AK1 was determined and expressed as a relative activity, relative to the enzyme activity obtained in the presence of NaCl—see FIG. 13. The pH optimum for AK1 is of the order of pH 6.9. As each imidazole compound gave distinctive inhibition of the enzymes. The inhibition of AK1 appears to be related to the protonation of the imidazole moiety, with AK1 being inhibited by the deprotonated form of the imidazole—FIG. 13. As the concentration of the protonated imidazole in solution increases with decreasing pH there is a concomitant increase in the activity of the enzyme. At a pH above pH 6.9 the deprotonated imidazole acts an inhibitor of AK1.

### Example 4: Effect of pH on the Inhibition of AK1 by Imidazole.HCl and Histidine.HCl and Deuterated Imidazole.HCl and Histidine.HCl.

The enzyme activity of AK1 was determined over a pH range of pH 6.3 to pH 8.1 in the presence of 2mM NaCl, imidazole.HCl and histidine.HCl and compared under the same conditions in the presence of deuterated imidazole.HCl and histidine.HCl—see FIG. 14. The imidazole.HCl and histidine.HCl was deuterated at position 5 which is equivalent to position C8 in the nucleotides. As outlined in Example 3, both imidazole.HCl and histidine.HCl inhibited the activity of AK1 when compared with the activity in the presence of NaCl. In both the case of imidazole.HCl and histidine.HCl there was a further reduction in the activity in excess of 50% of both adenylate kinases in the presence of deuterated imidazole.HCl and histidine.HCl.

Kinase inhibitors labeled at the carbon equivalent to C8 would therefore be at least double as inhibitory as unlabelled inhibitors.

### Example 5: Effect of Deuterated AMP and ATP on the Activity of AK1.

The enzyme activity of AK1 was determined using undeuterated nucleotides and compared with the activity in the presence of deuterated AMP, deuterated ATP and deuterated AMP and ATP. The assays contained 50 mM KH₂PO₄/K₂HPO₄ (at the equivalent pH), 0.6 mM ADP, 0.6 mM ATP and 0.66 mM MgCl₂.

The enzyme activity of AK1 was determined using undeuterated nucleotides and compared with the activity in the presence of deuterated ATP and undeuterated ATP (FIGS. 15 and 16). At low ATP concentrations AK1 the presence of undeuterated ATP gave lower activity than when deuterated ATP was used. At high ATP concentrations there is at least a 50% reduction in the activity as is the case in the presence deuterated ATP when compared with deuterated ATP.

### Example 6: The Effect of Adenosine 5′-[γ-thio]triphosphate (ATPS) and Deuterated ATPS on the Activity of AK1

The effect of ATPS and deuterated ATPS (C8-D ATPS) on the activity of AK1 was determined. Assays contained 1 nM human AK1, 0.66 mM MgCl₂, 0.6 mM ATP and 0.6 mM AMP in 50 mM potassium phosphate buffer, pH 7.2.

Increasing concentrations of ATPS or C8-D ATPS were added; 0.2, 0.4, 0.6, 0.8, and 1.0 mM. Assays, in a final volume of 1 ml, were allowed to proceed for 45 mins at 37° C., before 6 μl 100% TCA was added to stop the reaction. Both ATPS and C8-D ATPS were found to inhibit AK1; however the C8-D ATPS gave significantly more inhibition than the ATPS—see FIG. 17. The assay where no ATPS or C8-D ATPS was added produced 0.19 mM ADP.

### Example 7

AK1 was labeled by culturing the E. coli expression cells in the presence of ¹⁵N NH₄Cl and purified. A Heteronuclear Single Quantum Coherence (HSQC) ¹H and ¹⁵N NMR spectrum was obtained of AK1 in the presence of ATPS and C8-D ATPS at a concentration of ≅=200 μM AK1 and 100 mM of ATPS or C8-D ATPS. From the HSQC spectrum it is clearly evident that a number of the shifts in the active site of AK1 change their relative position as a result of the binding of the nucleotide in either the ATPS or C8-D ATPS forms—see FIG. 18).

A non-classical kinetic isotope effect is thus found in the enzyme kinetics of AK1 at low ATP concentrations, with a 50% reduction in enzyme activity, when the reactions are carried out using ATP when compared with ATP deuterated at position C8. A 50% reduction in enzyme activity is also obtained in the case where the kinase and synthase enzymes perform reversible reactions employing ADP or AMP.

All nucleotides bind into the active site of adenylate kinase enzymes by this mechanism which forms part of catalysis by inducing of C8-H to become more acidic.

A number of “imidazole” moiety-containing compounds inhibit adenylate kinase enzymes. A further reduction in enzyme activity by at least 50% is obtained when the “imidazole” moiety-containing compounds are deuterated at the position equivalent to C8 in AMP/ADP/ATP.

In accordance with the invention, a coupling mechanism is therefore provided whereby nucleotides bind to the active site of enzymes at the imidazole moiety by the inter-conversion of the C8-H. This coupling mechanism also plays a role in imidazole inhibition of adenylate kinases.

“Imidazole” moiety-containing compounds mimic the imidazole moiety of nucleotides and bond transiently but covalently to the active site of adenylate kinase enzymes in the same manner as nucleotides do.

### Example 8: The Effect of the ATP and C8D-ATP Concentration on the Specific Activity of Saccharomvces Cerevisiae Hexokinase, Escherichia Coli Acetate Kinase, Escherichia Coli Phosphofructokinase, Escherichia Coli Deadenylylated Glutamine Synthetase, Escherichia Coli Adenylylated Glutamine Synthetase and Mycobacterium Tuberculosis Shikimate Kinase

Based on the studies carried out on AK1 as presented in Examples 1 to 7, further comparative assays were run to determine the effect of ATP and C8D-ATP on the specific activity of a range of kinase enzymes. The kinase enzymes investigated were hexokinase, acetate kinase, shikimate kinase, phosphofructokinase, glutamine synthetase [(GS₁₂) and (GS₀)].

Acetate kinase (EC 2.7.2.1) is a homodimer which catalyses the Mg²⁺-dependent, reversible transfer of phosphate from ATP to acetate according to the following reaction:

CH₃COO⁻+ATP⇄CH₃CO₂PO₃²⁻+ADP

Acetate kinase forms part of the acetate and sugar kinase/Hsc70/actin (ASKHA) structural superfamily (PFam Clan: Actin ATPase:CL0108). The enzyme is a homodimer, and monomer interaction plays a role in the regulation of the enzyme activity and ligand binding with the enzyme active sites functioning in a coordinated half-the-sites manner. The acting ATPase clan contains both the acetate kinases and sugar kinases, and are all known to undergo a catalytically essential domain closure upon ligand binding.

Hexokinase (ATP: D-hexose 6-phosphotransferase, EC 2.7.1.1) catalyses the Mg²⁺-dependent phosphorylation of glucose, from ATP:

C₆H₁₂O₆+ATP⇄C₆H₁₂O₆PO₃²⁻+ADP

The two isoenzymes of yeast hexokinase, designated P-I and P-II, are dimers of subunit molecular mass 52 kDa. Hexokinase also forms part of the acetate and sugar structural superfamily (PFam Clan: Actin_ATPase:CL0108). Yeast hexokinase enzymes are structurally well characterised with each subunit of the homodimer comprising two domains and in the open conformation these domains are separated by a cleft containing the sugar binding sight. Binding of glucose induces a large conformational change in which the two lobes of the subunit rotate relative to each. The enzymes also exist in a monomer-dimer association-dissociation equilibrium that is influenced by pH, ionic strength and substrates. There are major differences in the glucose binding behaviour of both forms where binding to dimeric P-I shows strong positive cooperativity, whereas in P-II the two sites are equivalent and binding is non-cooperative.

Shikimate kinase. The shikimate pathway is a seven-step biosynthetic route that links the metabolism of carbohydrates to the synthesis of aromatic amino acids via the conversion of erythrose-4-phosphate to chorismic acid. Chorismic acid is an essential intermediate for the synthesis of aromatic compounds, such as aromatic amino acids, p-aminobenzoic acid, folate, and ubiquinone. Shikimate kinase (SK, EC 2.7.1.71), the fifth enzyme in the shikimate biosynthetic, catalyzes phosphate transfer from ATP to the carbon-3-hydroxyl group of shikimate, forming shikimate 3-phosphate. SK belongs to the nucleoside monophosphate (NMP) kinase structural family, with characteristic features of the NMP kinases being that they undergo large conformational changes during catalysis and belong to the P-loop containing nucleoside triphospahte hydrolase superfamily (Pfam Clan: AAA:CL0023). The NMP kinases are composed of three domains: the CORE, which contains a highly conserved phosphate-binding loop (P-loop); the LID domain, which undergoes substantial conformational changes upon substrate binding, and the NMP-binding domain, which is responsible for the recognition and binding of a specific substrate. MgADP induces concerted hinged movements of the shikimate binding and LID domains causing the two domains to move towards each other in the presence of this ligand. The SK crystal structures show that SK exists as a monomer with a single ATP binding site.

Phosphofructokinase (PFK, fructose-6-phosphate 1-kinase) (EC 2.7.1.11) is a classical allosteric enzyme that catalyzes the phosphorylation of D-fructose 6-phosphate (Fru-6-P) by Mg-ATP to form D-fructose 1,6-bisphosphate and MgADP. PFK from B. stearothermophilus is a homo-tetramer in which each subunit has a molecular weight of 34 000, and which undergoes a concerted two-state allosteric transition. PFK belongs to the PFK-like superfamily (Pfam Clan: PFK:CL0240) The enzyme from Bacillus stearothermophilus (Bs-PFK) shows hyperbolic Michaelis-Menten kinetics with respect to both Fru-6-P and Mg-ATP, but cooperative kinetics in the presence of allosteric inhibitor phosphoenolpyruvate(PEP). Unliganded Bs-PFK is in the active R state, which has high affinity for substrate, switching to the inactive T state with low affinity for substrate only in the presence of PEP.

Glutamine synthetase. Glutamine synthetase (GS) (EC 6.3.1.2) catalyzes the reversible conversion of L-glutamic acid, ATP and ammonia to L-glutamine, ADP and inorganic phosphate via a γ-glutamyl phosphate intermediate. As GS is a central enzyme in nitrogen metabolism the enzyme is regulated by at least four different mechanisms: (a) adenylylation and deadenylylation of the tyrosine 397 residue, (b) conversion between a relaxed (inactive) and taut (active) state depending on the divalent metal cation present, (c) cumulative feedback inhibition by multiple end products of glutamine metabolism, and (d) repression and derepression of GS biosynthesis in response to nitrogen availability. Escherichia coli GS is a large, metalloenzyme (˜624 kDa) comprising 12 identical subunits arranged in two face-to-face hexagonal rings. E. coli GS belongs to the glutamine synthetase 1-β group of enzymes that are regulated via adenylylation of a single tyrosine residue, with each subunit requiring two structurally implicated divalent cations (either Mg²⁺ or Mn²⁺) for its catalytic activity. The extent of adenylylation of the E. coli GS in response to an excess or deficiency of nitrogen in the growth environment is regulated in response to the intracellular concentrations of 2-ketoglutarate and glutamine, via the reversible adenylylation of a tyrosine residue (Tyr397) in each subunit of GS. The presence of adenylylated GS (GS₁₂) predominates in a nitrogen-rich, carbon-limited media, while the deadenylylated form (GS₀) tends to predominate under conditions of nitrogen limitation. The regulation of the adenylylation state of GS is accomplished by three proteins, uridylyltransferase/uridylyl-removing enzyme, the signal transduction protein P_{II}, and adenylyltransferase. High intracellular concentrations of glutamine activate the uridylyl-removing enzyme which causes the deuridylylation of P_{II}. This interacts with the adenylyltransferase which then catalyses the adenylylation of the GS. High intracellular concentrations of 2-ketoglutarate activate uridylyltransferase, which transfers UMP to each subunit of P_{II}, forming P_{II}-UMP. The P_{II}-UMP interacts with the adenylyltransferase, which in turn catalyses the removal of AMP from the GS.

Materials and Methods

Enzyme Source, and Protein Expression and Purification

Hexokinase from Saccharomyces cerevisiae Type F-300 (Sigma, H4502) and Acetate kinase from E. coli (Sigma, A7437) were purchased. The human adenylate kinase (AK1) gene in vector pLIG-SC1 was obtained from the Structural Genomics Consortium (code AK1A). The His-tagged AK1 was produced in Escherichia coli Origami (DE3) and purified using the Bio-Rad Profinia Purification System. The pure protein was dialysed against 50 mM KH₂PO₄/K₂HPO₄ buffer (pH 6.8), 1.5 mM MgCl₂, 120 mM KCl. The Mycobacterium tuberculosis shikimate kinase gene in pET15b (Novagen) was obtained from the group of Chris Abell, Cambridge University, UK. The his-tagged MtSK was produced in E. coli BL21 (DE3) and purified using the Bio-Rad Profinia Purification System. The pure protein was dialysed against 50 mM Tris (pH 7.5) and 1,000 mM NaCl. Adenylylated (GS₁₂) and deadenylylated (GS₀) glutamine synthetase were prepared as outlined below.

Production of glnD and glnE Knockout Strains

Knockout strains for the production of fully adenylylated (glnD knockout) or fully deadenylylated GS (glnE knockout) were made from the E. coli YMC11 using the Quick & Easy E. coli Gene Deletion Kit (Gene Bridges GmbH), designed to knockout or alter genes on the E. coli chromosome. Red/ET recombination allows the exchange of genetic information in a base pair precise and specific manner. An FRT-flanked kanamycin resistance marker cassette is supplied with the kit which can be used to replace a gene on the E. coli chromosome. The use of a FRT-flanked resistance cassette for the replacement of the targeted gene allows the subsequent removal of the selection marker by a FLP-recombinase step, involving the transformation of an FLP-expression plasmid into the cells and subsequent expression of an FLP site-specific recombinase. The genes for the Recombination proteins are under the control of an inducible promoter and the plasmid carries a temperature sensitive origin of replication for a convenient removal of the plasmid after recombination. In order to produce fully adenylylated GS, it is necessary to knockout the uridylyltransferase, coded by the glnD gene.

Primers were designed to the E. coli glnD gene. These primers contained a region specific to the glnD gene adjoining a sequence specific to the FRT cassette (underlined, see below). In a similar fashion, to produce fully deadenylylated GS, the adenylyltrasnferase, coded by the glnE gene, needs to be knocked out. Primers were therefore designed to the E. coli glnE gene. These primers contained a region specific to the glnE gene adjoining a sequence specific to the FRT cassette (underlined, see below). Both knockout strains were produced using the primers as described in the kit protocol. The only deviation from the protocol, was that BamHI restriction sites were incorporated in the ends of the primers (shown in bold). This enabled the PCR product to be cloned into pGEM T-Easy (Promega Corporation), and then cut out of the pGEM construct as a BamHI fragment. This facilitated production of the cassette in sufficient quantity for the transformation step, as it was found to be extremely difficult to produce enough of the cassette by PCR alone. Once integration of the cassette was confirmed by selection on kanamycin plates, a PCR product was produced using primers designed to the sequence of the glnD or glnE gene, either side of the integration site. This PCR product was then sequenced to confirm integration. The kanamycin resistance marker was removed using the 706-FLP plasmid carrying the site-specific recombinase. The removal of the marker was also confirmed by sequencing, as above. Primers used to create glnD and glnE knockout strains of E. coli YMC11 were:

glnD sense primer,
(SEQ ID NO. 56)
5′-gaggatcccagaaccagcgccatcagcgttaccatggcaccagc
tacaaccttgaaccaattaaccctcactaaagggcg-3′;
glnD antisense primer,
(SEQ ID NO. 57)
5′-gtggatccgcgatatcgtgaaacagcgcggcgatgaaaatcagc
tcagttgacggcagtaatacgactcactatagggctc-3′;
glnE sense primer,
(SEQ ID NO. 58)
5′-gaggatcctgcgcctgtttgaactgacgcagcgcctcaagctgt
tgctcttcgtcatcaattaaccctcactaaagggcg-3′;
glnE antisense primer,
(SEQ ID NO. 59)
5′-gtggatccaggtgttccagctcattcgcggcggacgcgaaccgt
cgctgcaatcgcgctaatacgactcactatagggctc-3′.

Purification of E. Coli Glutamine Synthetase

GS₁₂ and GS₀ were purified from recombinant E. coli YMC11 glnD and glnE knockout strains. E. coli YMC11 glnD strain producing GS₁₂ and the E. coli YMC11 glnE strain producing GS₀. The culturing protocols used were as outlined in supplementary information. The enzyme concentration and purity were determined by Quant -IT™ Protein Assay Kit (Invitrogen, USA) and SDS-PAGE.

C8-D ATP Synthesis.

The synthesis ATP and ADP deuterated at the C8 position (C8-D ATP and C8-D ADP) was carried out based on the method of (49). A 20 mM solution of Na₂ATP in D₂O containing 60 mM triethylamine (TEA) was incubated at 60° C. for 144 hours. The TEA was removed by twice passing the solution over a Dowex 20 W ion-exchange resin in the acid form. The pH of the solution was adjusted to pH 12 with NaOH prior to the second pass over the resin. The pH of the solution was adjusted to pH 6.3 prior to freeze drying. The extent of the deuteration of the C8 proton was determined by ¹H NMR and mass spectroscopy.

Steady-State Kinetic Analysis

GS₁₂, and GS₀ assay. The effect of the concentration of ATP and C8D-ATP on the specific activity of GS₁₂, and GS₀ was determined at concentrations ranging from 150 to 3000 μM ATP and C8D-ATP in assays containing 4 mM Na-glutamate, 4 mM NH₄Cl, 5.4 mM NaHCO₃ in 20 mM imidazole buffer. The GS₀ assay was carried out at pH 7.4 (±pH 0.05), and at MgCl₂ concentrations equivalent to 3 times the ATP concentration. The GS₁₂ assay was carried out at pH 6.6 (±pH 0.05), and at MnCl₂ concentrations equivalent to 3 times the ATP concentration. The reaction was stopped by the addition of tri-chloroacetic acid to give a pH of 2-3. The forward reaction rate was determined by measuring the ADP concentration in solution HPLC.

Hexokinase assay: 100 mM Phosphate buffer pH 6.8, 10 mM D-Glucose, 250 mM KCl, MgCl₂ and ATP were kept at a 1:1 ratio at concentration between 0.2 mM-3 mM. The assay was incubated at 37° C. for 15 minutes and stopped by the addition of 1 μl of 50% TCA. The formation of ADP was analysed by HPLC.

Acetatekinase assay: 100 mM Phosphate buffer pH 6.8, 10 mM Sodium Acetate, 250 mM KCl, MgCl₂ and ATP were kept at a 1:1 ratio at concentration between 0.2 mM-3 mM. The assay was incubated at 30° C. for 30 minutes and stopped by the addition of 1 μl of 50% TCA. The formation of ADP was analysed by HPLC.

Phosphofructokinase assay: 100 mM Phosphate buffer pH 6.8, 10 mM Fructose-6-Phosphate, 250 mM KCl, MgCl₂ and ATP were kept at a 1:1 ratio at concentration between 0.2 mM-3 mM. The assay was incubated at 37° C. for 15-30 minutes and stopped by the addition of 1 μl of 50% TCA. The formation of ADP was analysed by HPLC.

Shikimate kinase assay: Assays comprised 100 mM potassium phosphate buffer (pH 6.8), 500 mM KCl, 10 nM enzyme, and varying amounts of ATP, shikimic acid and MgCl₂. These were kept at a constant ratio of 1:1:2 for ATP: MgCl₂: shikimic acid. The ATP concentrations ranged between 0.2 and 10 mM. The final volumes were 100 μl, and the reactions were incubated at 37° C. for 20 minutes, before being terminated by the addition of 5 μl 200 mM EDTA.

In all assays, the production of ADP was analysed by HPLC.The assay solutions were centrifuged prior to HPLC analysis. The assays for adenosine, AMP, ADP ATP were carried out using Phenomenex 5p LUNA C₁₈ column with the mobile phase containing PIC A® (Waters Corporation), 250 ml acetonitrile, 7 g KH₂PO₄ per litre water. The flow rate of the mobile phase was 1 ml/minute with UV detection.

Results

ATP was deuterated specifically at position C8 as hereinbefore described and the deuteration was assessed by ¹H NMR.

The effect of the ATP and C8D-ATP concentration on the specific activity of Saccharomyces cerevisiae hexokinase, Escherichia coli acetate kinase, Escherichia coli phosphofructokinase, Escherichia coli deadenylylated glutamine synthetase, Escherichia coli adenylylated glutamine synthetase and Mycobacterium tuberculosis shikimate kinase was determined. The results are reflected in FIGS. 19 to 25. Where possible, the effect of the ATP and C8D-ATP on the specific activity of the enzyme was expressed over a concentration profile that included the ATP or C8D-ATP concentrations that would allow vₘₐₓ to be calculated as well as an ATP or C8D-ATP concentration profile at low concentrations that would allow for the accurate determination of the KIE. The best-fit to the data was obtained for the specified kinetic model using the non-linear regression algorithms as outlined using the GraphPad Prism® 5 software. As part of the software output, a data-table was created containing 150 data-points defining the best kinetic fit for each enzymes response to the presence of either ATP or C8D-ATP (see table 2 for kinetic model). These response curves were then used to define the KIE by the conventional estimation of KIE from KIE=v_{H}/v_{D}. The KIE_{D} was also determined using the following function:

$${KIE}_{D} = \frac{{v}_{D}}{{v}_{H}}$$

Where v_{D}=specific activity in the presence of C8D-ATP
v_{H}=specific activity in the presence of ATP.

The calculation of KIE_{D} was used as the data obtained is instructive in a putative role that the C8H of ATP plays in the regulation of phosphoryl transfer.

In all 6 cases defined:
A KIE was obtained in response to presence of C8D-ATP (FIGS. 19-24).
In all cases other than shikimate kinase, the KIE_{D} at low ATP concentrations is in excess of 5 (FIG. 25).
In monomeric enzymes, such as shikimate kinase, as the concentration of ATP and C8D-ATP was increased, there was a concomitant increase in the KIE_{D} while in oligomeric enzymes a there was a decrease in the KIE_{D} (FIG. 19).
In all cases the KIE obtained was a primary KIE as extent of the KIE was two-fold or significantly in excess of two-fold at low concentrations.
The KIE_{D} over the full ATP/C8D-ATP concentration range appeared to be indicative of the mode of regulation of the enzyme as in all cases the KIE either positively or negatively asymptotes to a specific constant value.
The KIE of shikimate kinase asymptotes positively to a KIE of 1.0 as the specific activity tends towards vₘₐₓ. The KIE giving a classical KIE effect with the KIE being 2 at low ATP concentrations asymptoting to a level of 1 (FIG. 19, Table 2). Shikimate kinase exists as a monomer and therefore no regulation occurs via the interaction of the subunits that may affect the overall KIE.
Hexokinase, acetate kinase and GS₀ use the same mechanism for regulation. The KIE_{D} of these enzymes negatively asymptote to 1 at Vₘₐₓ (FIGS. 20-22, Table 2). All three of these enzymes are multi-meric and allosteric regulation may occur via the interaction of sub-units. The hexokinase and acetate kinase are both homodimers and monomer interaction plays a role in the regulation of the enzyme activity and ligand binding with the enzyme active sites functioning in a coordinated half-the-sites manner.
Phosphofructokinase and GS₁₂ use a similar mechanism with the KIE_{D} asymptoting to a level of 0.5 at vₘₐₓ (KIE=2) (FIGS. 22-24, Table 2). E. coli GS₁₂ is a dodecamer consisting of two stacked hexameric structures consisting of 12 identical subunits. The subunits probably interact allosterically on the binding of ATP as occurs in phosphofructokinase. The slow rate of release of C8D-ADP from the interacting active site of GS₁₂ probably impacting on the binding of ATP in the adjacent site.

TABLE 2
Effect of the concentration of ATP and C8D-ATP on the fit of the enzyme kinetic model of hexokinase, acetate
kinase, adenylylated GS, deadenylylated GS and shikimate kinase. The response of each enzyme to change
in the ATP and C8D-ATP concentration was tested for the fit to either an allosteric sigmoidal model or to the
Michaelis-Menton model of enzyme kinetics by non-linear regression using the GraphPrism 5 software. The
root mean square deviation of the data from the model is as outlined. The Hill factor for the allosteric sigmoidal
model is as indicated. KIEᵥₘₐₓ is equal to the KIE attained at ATP and C8D-ATP concentrations at
maximum enzyme activities.

| | | ATP fit to kinetic | C8D-ATP fit to | | | RMSD^{a} | | |
|---|---|---|---|---|---|---|---|---|
| Enzyme | Kinetic Model | model | kinetic model | h_{ATP} | h_{C8D} | ATP | C8D | KIEᵥₘₐₓ |
| Hexokinase | Allosteric sigmoidal | Allosteric sigmoidal | Fit ambiguous | 1.75 | | 0.9963 | | 1 |
| | Michaelis-Menton | Fit ambiguous | Michaelis-Menton | | | | 0.9885 | |
| Acetate kinase | Allosteric sigmoidal | Allosteric sigmoidal | Fit ambiguous | 2.08 | | 0.9865 | | 1 |
| | Michaelis-Menton | Fit ambiguous | Michaelis-Menton | | | | 0.9847 | |
| Dedenylylated GS | Allosteric sigmoidal | Allosteric sigmoidal | Allosteric sigmoidal | 3.10 | 4.08 | 0.9972 | 0.9827 | 1 |
| | Michaelis-Menton | Fit ambiguous | Fit ambiguous | | | | | |
| PFK^{b} | Allosteric sigmoidal | Allosteric sigmoidal | Allosteric sigmoidal | 1.37 | 1.79 | 0.9982 | 0.9879 | 2 |
| | Michaelis-Menton | Fit ambiguous | Fit ambiguous | | | | | |
| Adenylylated GS | Allosteric sigmoidal | Allosteric sigmoidal | Allosteric sigmoidal | 1.77 | 0.92 | 0.9940 | 0.9990 | 2 |
| | Michaelis-Menton | Fit ambiguous | Fit ambiguous | | | | | |
| Shikimate kinase | Allosteric sigmoidal | Allosteric sigmoidal | Allosteric sigmoidal | 1.22 | 1.06 | 0.9870 | 0.9770 | 1 |
| | Michaelis-Menton | Fit ambiguous | Fit ambiguous | | | | | |

^{a}Root mean square deviation of the data defining the kinetic model.
^{b}Phosphofructokinase

Discussion

The role of the KIE in the kinetics of the enzymes investigated lead to models for the regulation of the binding of ATP being proposed, as set out in FIG. 25.

In classical steady-state kinetics as represented by the Briggs-Haldane modification of the Michael-Menton formulation (Equation 1),

$$E + S {{{←}_{\to }}_{{k}_{on}}}^{{k}_{off}} ES {\to }^{{k}_{cat}} E + P \qquad ( Eq . 1 )$$

and kₒₙ=k₁, k_{off}=k₋₁ and k_{cat}=k₂, k₂>>k₋₁, and the Michaelis constant, K_{M} is obtained from

$$\frac{[ E ] [ S ]}{[ ES ]} = \frac{{k}_{- 1} + {k}_{2}}{{k}_{1}} = {K}_{M} \qquad ( Eq . 2 )$$

In monomeric enzymes such a shikimate kinase K_{M} is dependent only on k₂. The effect of the increase in the ATP/C8D-ATP concentration on the KIE therefore only manifests as the classical effect with the KIE being of the order of 2.0 as determined by v_{H}/v_{D}, at low concentrations, asymptoting to 1 at high ATP concentrations. At low concentrations of ATP the enzyme activity is dominated by the impact of the C8H/C8D on the equilibrium of binding. At high ATP concentrations the impact of the increase in the ATP concentration on the equilibrium overrides the effect of the C8H/C8D on binding resulting in a decrease in the KIE. As the classical H/D KIE is of the order of 2, as the concentration of ATP tends towards the concentration at the maximum specific activity, vₘₐₓ, where the concentration effect is at its maximum the effect of the C8H/C8D on the KIE is at a minimum and the KIE tends towards 1.

In oligomeric enzymes it is proposed that the deuteration of ATP not only affects the binding of ATP to the site where catalysis is occurring but the deuteration also affects the interaction between sites. In oligomeric kinases it is proposed that mechanistically two modes of regulation occur, one which is dependent on the release of ADP from the first active site before ATP binds to the second active site (FIG. 26B) and the second mode of regulation depends on the conversion of ATP to ADP prior to the binding of the ATP to the second active site (FIG. 26C). In the mechanism outlined in FIG. 25C binding to the second site can occur prior to the release of ATP from the first site.

It is proposed that in enzymes such as acetate kinase, hexokinase and GS₁₂ the enzyme kinetics follows classical Michaelis-Menton kinetics where an equilibrium is set up between the enzyme concentration [E] and the substrate concentration [S] and binding of the second ATP is dependent on the conversion of the second active site into an ATP binding form by the release of ATP from the first active site, as defined by the coordinated half-sites mechanism. In enzymes using this mechanism of regulation, K_{M} is dependent on k₋₁ and k₂. The KIE obtained in these enzymes asymptotes to a value of 1. At low ATP concentrations the effect of the deuteration of C8 is to allow binding to occur for long enough to allow the reaction to occur and negate the effect of k₋₁, thereby shifting the equilibrium to k₂. At low ATP concentrations therefore the impact of the deuteration on the binding is to retard the release of the ATP. At high ATP concentrations the impact of the ATP concentration relative to the impact of ATP binding on the rate of reaction is significantly higher and as a result there is a concomitant increase in the KIE. The impact of binding and the reaction rate however equilibrate to a KIE of 1. The maximum rate of binding can only ever be equivalent to the maximum rate at which the second ATP binding site is converted to the ATP binding form by the release of ATP from the first site (FIG. 26B). The classical impact of deuteration on the KIE when the KIE is a primary effect, as determined by v_{H}/v_{D}, should yield a KIE of 2 or more. As the regulation of the enzyme activity and ligand binding in these enzymes function in a coordinated half-the-sites manner binding in the second site only occurs on release of the ADP from the first site, it is therefore proposed that deuteration of the ATP improves the binding characteristics. As the equilibrium shifts towards the impact of increasing ATP concentration on the enzyme activity the deuterated ATP binds effectively twice as efficiently as the non-deuterated ATP thereby negating the impact of the deuteration on the apparent enzyme activity at high ATP concentrations, yielding a KIE of 1.

In enzymes where the second active site is made amenable to ATP binding by the conversion of ATP to ADP, in other words binding may occur to the second site prior to the release of the ATP from the first site, the K_{M} is dependent on k₁ and k₂. This occurs in the case of phosphofructokinase and GS₁₂ where the KIE becomes 2 at vₘₐₓ (FIGS. 23 & 24). The impact of this binding is that at any point in time up to two or more reactions might be occurring simultaneously in two active sites. In multi-meric enzymes this effect might be greater. As the deuterated ATP binds twice as efficiently as the non-deuterated ATP this allows the KIE to asymptote to 2 or more. It is proposed that a result of the adenylylation of GS it allows for the regulation of the enzyme by a similar mechanism as occurs in phosphofructokinase. Bacterial PFK is a homoteramer, with the four subunits assembled as a dimer of dimers. It is conceivable that on adenylylation of GS the interaction between two-subunits effectively creates a dimer of dimer interaction.

The data outlined clearly demonstrates the role of C8H of ATP in the kinetics of a number of kinase and synthetase enzymes. The KIE is clearly a primary KIE however the extremely high values of the KIE obtained at low at concentrations in the case of the oligomeric enzymes does not appear to be as a result of the impact of the deuterium on the rate the phosphoryl transfer mechanism per se but rather as a result of the role that the C8H plays in the equilibrium of binding of the ATP to the active site (FIG. 25). Clearly the regulation of enzyme activity in kinases and synthetases is complex which manifests in the apparent Kₘ of the kinases ranges from less than 0.4 μM to in excess of 1000 μM for ATP (Carna Biosciences, Inc., Kinase Profiling Book:www.carnabio.com). The findings of this investigation have discovered that the C8H of ATP plays a direct role in binding of ATP to the active site of enzymes.

The deuteration of compounds containing imidazole moieties that are currently used as drugs, will increase their efficacy. With the increase in the efficacy of the deuterated forms of current drugs containing imidazole moieties either in use or in clinical trails, dosage levels of these compounds may be reduced to alleviate the toxicity.

1. A method of inhibiting the activity of a kinase or a synthetase, the method including binding an active site of the kinase or synthetase with a deuterated imidazole moiety, thereby inhibiting the activity of the kinase or the synthetase.

2. The method of claim 1, wherein it is applied to a kinase or synthetase, and wherein the kinase is selected from the group consisting of adenylate kinase (AK), shikimate kinase (SK), pyruvate kinase (PK), hexokinase (HXK), aspartokinase (ASK), creatine kinase (CK), glycerate kinase, acetate kinase, phosphofructokinase and the synthetase is glutamine synthetase, such as adenylated glutamine synthetase, and/or deadenylated glutamine synthetase, and isoforms thereof.

3. The method of claim 2, wherein the kinase is adenylate kinase, whose sequence comprises conserved residues Arg 97, Glu 98, Arg 128 and Asp 180.

4. The method of claim 1, wherein the kinase or the synthetase, or an isoenzyme thereof, comprises amino acid residues identical to, or similar to, conserved adenylate kinase residues Arg 97, Glu 98, Arg 128 and Asp 180, at positions equivalent to the positions of these residues in adenylate kinase.

5. The method of any one of claim 1, wherein the inhibition of the kinase or the synthetase activity is effected in vitro.

6. The method of claim 1, wherein the deuterated imidazole moiety is provided by a nucleotide.

7. The method of claim 6, wherein the nucleotide is adenosine triphosphate (ATP), adenosine diphosphate (ADP) or adenosine monophosphate (AMP), with the nucleotide having the C8-H induced to be more acidic, and with deuteration being effected at the C8 position.

8. The method of claim 6, wherein the nucleotide is a compound which is deuterated at a position equivalent to C8 in ATP, ADP and AMP.

9. A method of coupling a kinase or a synthetase to a nucleotide or to a nucleotide analogue, to inhibit the activity of the kinase or the synthetase, the method including binding an active site of the kinase or the synthetase with a nucleotide or with a nucleotide analogue comprising an imidazole moiety in deuterated form.

10. A method of generating a compound that inhibits the activity of a kinase or a synthetase, the method comprising providing a three-dimensional structure of a kinase or a synthetase; and designing, based on the three-dimensional structure, a compound capable of inhibiting the activity of the kinase or the synthetase, the compound comprising a deuterated imidazole moiety.

11. A computer-assisted method of generating a test inhibitor of the activity of a kinase or a synthetase, the method using a processor and an input device, the method comprising
(a) inputting, on the input device, data comprising a structure of a kinase or a synthetase;
(b) docking into an active site of the kinase or the synthetase, a test inhibitor molecule comprising a deuterated imidazole moiety, using the processor; and
(c) determining, based on the docking, whether the test inhibitor compound would inhibit the kinase or synthetase activity.

12. The method of claim 11, which includes determining, based on the docking, whether the test inhibitor molecule would inhibit the transfer of a γ-phosphate group from a phosphate donor.

13. The method of claim 11, which further comprises designing a test inhibitor determined by step (c) to inhibit the kinase or the synthetase activity and evaluating the inhibitory activity of the test inhibitor on a bacterial kinase or synthetase in vitro.

14. A method of screening a compound in vitro to determine whether or not it inhibits the activity of kinase or a synthetase, the method comprising contacting a kinase or a synthetase with a compound comprising a protonated imidazole moiety; contacting the kinase or the synthetase with the same compound comprising a deuterated imidazole moiety;
and determining whether or not the activity of the kinase or synthetase is reduced in the presence of the compound containing the deuterated imidazole moiety relative to the activity of the same kinase or synthetase in the presence of the compound containing the protonated imidazole moiety.

15. The method of claim 9 wherein it is applied to a kinase, and, wherein the kinase is selected from a group consisting of adenylate kinase (AK), shikimate kinase (SK), pyruvate kinase (PK), hexokinase (HXK), aspartokinase (ASK), creatine kinase (CK), glycerate kinase, acetate kinase, phosphofructokinase, and the synthetase is glutamine synthetase, such as adenylated glutamine synthetase, and/or deadenylated glutamine synthetase, and isoforms thereof.

16. The method of claim 9, wherein the deuterated imidazole moiety is provided by a nucleotide or by a nucleotide analogue.

17. The method of claim 16, wherein the nucleotide is adenosine triphosphate (ATP), adenosine diphosphate (ADP) or adenosine monophosphate (AMP), with the nucleotide having an immonium moiety at position N7, so that a carbene is induced at the C8 position, and with deuteration being effected at the C8 position.

18. The method of claim 16, wherein the nucleotide is a compound which is deuterated at a position equivalent to C8 in ATP, ADP and AMP.
